(19) United States
(12) Patent Application Publication
(10) Pub. No.: US 2020/0319203 A1
(43) Pub. Date: Oct. 8, 2020

(54) COMPOSITIONS AND METHODS FOR FETAL GROWTH

(71) Applicant: The Board of Supervisors of Louisiana State University and Agricultural and Mechanical College, Baton Rouge, LA (US)

(72) Inventors: Leanne Maree Redman, Zachary, LA (US); Elizabeth Frost Sutton, Baton Rouge, LA (US)

(21) Appl. No.: 16/772,708

(22) PCT Filed: Dec. 19, 2018
(86) PCT No.: PCT/US2018/066439

§ 371 Date: Jun. 12, 2020

Related U.S. Application Data
(60) Provisional application No. 62/607,614, filed on Dec. 19, 2017.

(51) Int. Cl. G01N 33/68; C07K 16/22; G01N 33/543

(52) U.S. Cl. CPC G01N 33/6872; C07K 16/22; G01N 2800/368; G01N 2333/50; G01N 33/543

(57) ABSTRACT

This invention is directed to methods and compositions for detecting fetal growth restriction

This application claims priority from U.S. Provisional Application No. 62/607,614, filed on Dec. 19, 2017, the entire contents of which is incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under Grant No. U01DK094418, Grant No. R01DK099175 and Grant No. F31HD084199 awarded by the National Institutes of Health. The government has certain rights in the invention.

### FIELD OF THE INVENTION

This invention is directed to methods and compositions for detecting fetal growth restriction.

### BACKGROUND OF THE INVENTION

Intrauterine Growth Restriction (IUGR), also known as Fetal Growth Restriction, is when a fetus in the womb fails to grow at the expected rate during the pregnancy. In other words, at any point in the pregnancy, the baby is not as big as would be expected for how far along the mother is in her pregnancy (this timing is referred to as an unborn baby's “gestational age”).

Babies who have fetal growth restriction often have a low weight at birth. If the weight is below the 10th percentile for a baby's gestational age (meaning that 90% of babies that age weigh more) the baby is also referred to as “small for gestational age,” or SGA.

IUGR can be classified as symmetrical IUGR, in which a baby's body is proportionally small (meaning all parts of the baby's body are similarly small in size), or asymmetrical IUGR, which is when the baby has a normal-size head and brain but the rest of the body is small.

Fundal height, i.e. measurement of the size of the uterus across a patient's abdomen with a tape measure, is currently the only routine method employed in the clinical setting for detection of fetal growth restriction or retardation (of note, ultrasound imaging may be implemented if a patient has a higher risk or predisposition to an IUGR fetus). When considering the potential consequences of growth restriction for a fetus, including fetal mortality, the need for more reliable assessments of growth restriction to supplement this archaic and highly unreliable assessment is apparent.

### SUMMARY OF THE INVENTION

The invention provides a non-invasive method of identifying a fetus at risk for fetal growth restriction. In embodiments, the method comprises incubating a biological sample from a subject pregnant with a fetus with an agent that binds to FGF21 protein or a fragment thereof, wherein a FGF21-binding agent complex is formed; measuring the amount of FGF21 bound agent in the biological sample obtained from the subject; and identifying the fetus as at risk for fetal growth restriction when FGF21 protein levels in the sample are elevated above control levels.

The invention further provides for a non-invasive method to identify a fetus with fetal growth restriction. In embodiments, the method comprises incubating a biological sample from a subject pregnant with a fetus with an agent that binds to FGF21 protein or a fragment thereof, wherein a FGF21-binding agent complex is formed; measuring the amount of FGF21 bound agent in the biological sample obtained from the subject; and identifying the fetus as having fetal growth restriction when FGF21 protein levels in the sample are elevated above control levels.

Still further, the invention provides for a non-invasive method of identifying a fetus exposed to nutrient insufficiency. In embodiments, the method comprises measuring the level of FGF21 or a fragment thereof in the sample obtained from a subject pregnant with the fetus, wherein a level of FGF21 in the sample elevated above control levels identifies a fetus exposed to nutrient insufficiency.

In embodiments, measuring comprises incubating the biological sample with an agent that binds to FGF21 protein or a fragment thereof, wherein a FGF21-binding agent complex is formed. In embodiments, the FGF21 binding agent comprises an anti-FGF21 antibody.

In embodiments, the biological sample comprises serum, whole blood, plasma, or a combination thereof.

In embodiments, measuring comprises an immunoassay, a colorimetric assay, a fluorimetric assay or a combination thereof. For example, the immunoassay comprises a Western blot assay, an enzyme-linked immunosorbent assay (ELISA), immunoprecipitation or a combination thereof.

The invention also provides a method of treating fetal growth restriction. In embodiments, the method comprises administering an amount of protein sufficient to reduce circulating FGF21 levels in a subject pregnant with a fetus determined to be afflicted with fetal growth restriction, wherein fetal growth restriction is determined by measuring FGF21 levels in a biological sample isolated from the pregnant mother.

Further, the invention provides a method of preventing fetal growth restriction. In embodiments, the method comprises administering a sufficient amount of a protein to a subject pregnant with a fetus at risk of fetal growth restriction to reduce circulating FGF21 levels, wherein risk of fetal growth restriction is determined by measuring FGF21 levels in a biological sample isolated from the pregnant mother.

Embodiments can further comprise comparing the level of FGF21 protein in the sample to that of at least one control sample.

Embodiments can further comprise administering to the pregnant mother an amount of protein sufficient to reduce or restore circulating FGF21 levels. In embodiments, the amount of protein sufficient to reduce or restore circulating FGF21 levels comprises no less than about 0.88 g/kg of body weight/day.

The invention provides a method of treating fetal growth restriction. In embodiments, the method comprises administering to a subject pregnant with a fetus afflicted with or at risk of fetal growth restriction a therapeutically effective amount of an agent that reduces the circulating protein level of FGF21.

Embodiments further comprise determining the risk for fetal growth restriction by measuring the level of FGF21 protein or a fragment thereof in a biological sample isolated from the subject.

In embodiments, measuring comprises incubating the biological sample with an agent that binds to FGF21 protein or a fragment thereof, wherein a FGF21-binding agent complex is formed. In embodiments, the FGF21 binding agent comprises an anti-FGF21 antibody.

In embodiments, the biological sample comprises serum, whole blood, plasma, or a combination thereof.

In embodiments, measuring comprises an immunoassay, a colorimetric assay, a fluorimetric assay or a combination thereof. For example, the immunoassay comprises a Western blot assay, an enzyme-linked immunosorbent assay (ELISA), immunoprecipitation or a combination thereof.

In embodiments, the agent is administered to the placenta. Non-limiting examples of agents comprise a synthetic polynucleotide, such as a synthetic polynucleotide that is targeted to the nucleic acid molecule encoding FGF21 as in NCBI reference sequence number NM 019113 (SEQ ID NO: 3). In embodiments, the synthetic polynucleotide comprises siRNA.

In embodiments, FGF21 comprises intact FGF21 (SEQ ID NO: 6).

In embodiments, the FGF21 fragment comprises N-terminal truncated FGF21 (7-181) (SEQ ID NO:5), FGF21 truncated at the N-terminus by 4 amino acids (SEQ ID No. 7).

The invention further provides a kit of molecular biomarkers for identifying a fetus at risk for growth restriction. In embodiments, the kit comprises at least one element for measuring the level of FGF21 protein or fragment thereof, and, optionally, at least one element for measuring the level of β-Klotho, where together represent a molecular signature that is indicative of fetal growth restriction.

In embodiments, the signature of fetal growth restriction comprises levels of FGF21 and, optionally, β-Klotho, above control levels.

Other objects and advantages of this invention will become readily apparent from the ensuing description.

### BRIEF DESCRIPTION OF THE FIGURES

FIG. 1 shows FGF21 levels in 1st and 3rd trimester subjects, demonstrating that FGF21 increases across pregnancy. FGF21 concentrations in the first and third trimesters of our study population. Individual and mean±SEM serum FGF21 measured across pregnancy, 1^{st} trimester: 0.105±0.01 ng/mL, 3^{rd} trimester: 0.248±0.03 ng/mL, *p<0.0001.

FIG. 2 shows FGF21 positively correlates with BMI and fat mass in pregnancy. Serum FGF21 is significantly correlated with maternal body mass index but not body weight throughout pregnancy. (Panel A) 1^{st} trimester BMI, n=29, r=0.48, p=0.008, (Panel B) 3^{rd} trimester BMI, n=43, r=0.38, p=0.01, (Panel C) 1^{st} trimester body weight, n=29, r=0.34, p=0.07, and (Panel D) 3^{rd} trimester body weight, n=43, r=0.19, p=0.23.

FIG. 3 shows rise in FGF21 correlates with decreasing in fasting glucose across pregnancy. The correlation between % change FGF21 and % change glucose. Change in FGF21 across pregnancy is correlated with change in maternal fasting glucose but not change in maternal body size or adiposity. Percent change in FGF21 from first to third trimester compared to (Panel A) percent change in body weight (n=29, r=0.12, p=0.52), (Panel B) percent change in fat mass (n=29, r=0.02, p=0.93), (Panel C) percent change in fat free mass (n=29, r=0.18, p=0.35), (Panel D) percent change in glucose (n=29, r=−0.40, p=0.03), (Panel E) percent change in insulin (n=29, r=−0.23, p=0.22), and (Panel F) percent change in HOMA-IR (n=29, r=−0.28, p=0.14).

FIG. 4 shows serum FGF21 levels in 3rd trimester subjects with low protein, normal protein, or high protein diets. Serum FGF21 is elevated in states of low protein intake during pregnancy in humans. Serum FGF21 concentrations from humans during 35-36 weeks gestation self-selecting low (0.302±0.163 ng/mL), normal (0.206±0.155 ng/mL) or high (0.133±0.061 ng/mL) protein diets, *p=0.04.

FIG. 5 shows maternal FGF21 is negatively correlated with fetal and infant growth. Maternal third trimester FGF21 concentration is negatively correlated with infant length at birth. (Panel A) Maternal 3^{rd} trimester FGF21 and infant birth length (n=27, p=0.03, r=−0.45) and (Panel B) maternal 3^{rd} trimester FGF21 and infant birth length percentile (n=27, p=0.02, r=−0.46). (Panel C) Infant weight, grams (4-8 weeks old), (Panel D) infant length, cm (4-8 weeks old).

FIG. 6 shows maternal FGF21 is negatively correlated with infant head circumference in the 1st year of life. Maternal third trimester FGF21 concentration is negatively correlated with infant head circumferences through the first year of life. (Panel A) Maternal 3^{rd} trimester FGF21 and infant head circumference (n=25, r=−0.66, p<0.01) and (Panel B) percentile (n=25, r=−0.45, p=0.03) at 4-8 weeks of age, (Panel C) maternal 3^{rd} trimester FGF21 and infant head circumference (n=24, r=−0.53, p=0.01) and (Panel D) percentile (n=24, r=−0.58, p=0.01) at 6 months of age, and (Panel E) maternal 3^{rd} trimester FGF21 and infant head circumference (n=24, r=−0.66, p<0.01) and (Panel F) percentile (n=24, r=−0.66, p<0.01) at 1 year of age.

FIG. 7 shows device for measuring food intake by mice (see Sorensen, Allan, et al. “Protein-leverage in mice: the geometry of macronutrient balancing and consequences for fat deposition.” Obesity 16.3 (2008): 566-571).

FIG. 8 shows protein intake by wildtype C57BL6 mice and indicates eating pattern of wildtype C57BL6 mice. Diets provided: 4% protein, 36% protein or 55% protein; Daily protein intake from 3 individual mice representative of each group (20 mice per group).

FIG. 9 shows protein intake by FGF21 knockout mice and indicates eating pattern of FGF21 knockout mice. Female FGF21KO mice are incapable of regulating protein intake. Diets provided: 4% protein, 36% protein, or 55% protein; Daily protein intake from 3 individual mice representative of each group (19 mice per group).

FIG. 10 shows comparison of average protein intake by FGF21 knockout mice (n=19) and wildtype mice (n=20).

FIG. 11 shows analysis of significance.

FIG. 12 shows schematic of research protocol.

FIG. 13 shows food intake by pregnant C57BL/6 wildtype mice.

FIG. 14 shows food intake by pregnant FGF21 knockout mice.

FIG. 15 shows protein leverage in response to increased protein demand of pregnancy.

FIG. 16 shows FGF21 increases across pregnancy. Serum FGF21 concentrations in the first and third trimesters of the study population. (Panel A) Distribution of FGF21 in the 1^{st} trimester (<16 weeks) (n=29), (Panel B) Distribution of FGF21 in the 3^{rd} trimester (35-36 weeks) (n=43), (Panel C) Individual and mean±SEM serum FGF21 measured across pregnancy, 1^{st} trimester: 0.105±0.01 ng/mL, 3^{rd} trimester: 0.248±0.03 ng/mL, *p<0.0001.

FIG. 17 shows FGF21 did not correlate with glucose homeostasis in pregnant cohort. First and third trimester serum FGF21 is not correlated with maternal glucose homeostasis in early or late pregnancy. (Panel A) 1^{st} trimester glucose and FGF21, n=29, r=0.12, p=0.55, (Panel B) 1^{st} trimester insulin and FGF21, n=29, r=0.26, p=0.18, (Panel C) 1^{st} trimester HOMA-IR and FGF21, n=29, r=0.25, p=0.20, (Panel D) 3^{rd} trimester glucose and FGF21, n=42, r=0.26, p=0.10, (Panel E) 3^{rd} trimester insulin and FGF21, n=42, r=0.11, p=0.48, and (Panel F) 3^{rd} trimester HOMA-IR and FGF21, n=42, r=0.14, p=0.38.

FIG. 18 shows FGF21 is elevated in pregnant wild-type mice consuming low protein diet in gestation. Serum FGF21 concentrations from C57BL/6 mice on day 18.5 gestation fed low (7%) or normal (21%) protein diets, (22.760 ±7.416 ng/mL vs. 1.673±0.176 ng/mL, *p=0.006, n=5-7).

FIG. 19 shows protein restriction increases food intake in pregnant mice, but this effect is lost in FGF21 knockout mice.

FIG. 20 shows (Panel A) final pregnant body weight, (Panel B) uterine weight and (Panel C) body weight minus uterine weight (residual maternal weight). Protein restriction reduces BW and uterine weight in both mouse lines. However, the effect on BW is smaller and on uterine weight slightly bigger in the KO mice. As a result, residual BW (body weight minus uterine weight) is not reduced in the FGF21-KO moms. Without wishing to be bound by theory, FGF21-KO moms do not sacrifice their own body weight to support the pregnancy the way wildtype mice do.

FIG. 21 shows final pregnant body weight (BW), uterine weight (UW) and body weight minus uterine weight (residual maternal weight; BW-UW).

FIG. 22 shows serum FGF21 concentrations in the first and third trimesters of our study population. (Panel A) Distribution of FGF21 in the 1st trimester (<16 weeks), (Panel B) distribution of FGF21 in the 3rd trimester (35-36 weeks), (Pabel C) individual and mean±SEM serum FGF21 measured across pregnancy, (Panel D) individual change in serum FGF21 between first and third trimesters, *p<0.0001.

FIG. 23 shows Serum FGF21 is significantly correlated with maternal body mass index and fat mass throughout pregnancy. (A) 1st trimester BMI, (B) 3rd trimester BMI, (C) 1^{st} trimester fat mass, (D) 3^{rd} trimester fat mass.

FIG. 24 shows (Panel A) first trimester fat mass, (Panel C) first trimester fat free mass, (Panel B) third trimester fat mass, and (Panel D) third trimester fat free mass.

FIG. 25 shows change in (Panel A) weight, (Panel D) glucose, (Panel B) fat mass, (Panel E) insulin, (Panel C) fat free mass, and (Panel F) HOMA-IR relative to change in FGF21.

### DETAILED DESCRIPTION OF THE INVENTION

Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Fibroblast Growth Factor 21

Fibroblast Growth Factor 21 (FGF21) is a member of the “endocrine” FGF family, and belongs to the FGF-19 subfamily, which includes FGF-19, FGF21 and FGF-23. The FGF-19 family members are potent endocrine hormones in the regulation of a diverse physiological homeostasis.

Fibroblast growth factor 21 (FGF21) is a 19 kDa, 209 amino acid long peptide produced by the liver, white and brown adipose tissue, muscle (smooth and skeletal), thymus, and endocrine and exocrine pancreas in humans. During pregnancy, the placenta does not contribute significant amount of FGF21 to the maternal circulation.

FGF21 is a member of the “endocrine” subgroup of the fibroblast growth factor family, along with FGF19 and FGF23, grouped together based on their similar structural homology and ability for endocrine action. FGF21 was discovered in 2000. Murine FGF21 has 75% homology with human FGF21 and 35% homology with human FGF19. At the time of its identification, FGF21 was reported to be preferentially expressed by the liver (Nishimura, T. et al, 2000. Identification of a novel FGF, FGF21, preferentially expressed in the liver. Biochimica et biophysica acta 1492:203-206.). Five years later, FGF21 was revealed to have an adipocyte-specific glucose sensitizing capability (Kharitonenkov, A., et al 2005. FGF21 as a novel metabolic regulator. The Journal of clinical investigation 115:1627-1635).

FGF21 is produced by the liver (Nishimura, T., et al. 2000. Identification of a novel FGF, FGF21, preferentially expressed in the liver. Biochimica et biophysica acta 1492:203-206.; Fon Tacer, K., et al. 2010. Research resource: Comprehensive expression atlas of the fibroblast growth factor system in adult mouse. Molecular endocrinology 24:2050-2064; Cui, Y., et al. 2014. Hepatic FGF21 production is increased in late pregnancy in the mouse. American journal of physiology. Regulatory, integrative and comparative physiology 307:R290-298; Markan, K. R., et al. 2014. Circulating FGF21 is liver derived and enhances glucose uptake during refeeding and overfeeding. Diabetes 63:4057-4063), white adipose tissue (Zhang, X., et al. 2008. Serum FGF21 levels are increased in obesity and are independently associated with the metabolic syndrome in humans. Diabetes 57:1246-1253; Fon Tacer, K., et al. 2010. Research resource: Comprehensive expression atlas of the fibroblast growth factor system in adult mouse. Molecular endocrinology 24:2050-2064; 116), brown adipose tissue (Fon Tacer, K., et al. 2010. Research resource: Comprehensive expression atlas of the fibroblast growth factor system in adult mouse. Molecular endocrinology 24:2050-2064; Hondares, E., et al. 2011. Thermogenic activation induces FGF21 expression and release in brown adipose tissue. The Journal of biological chemistry 286:12983-12990; Hondares, E., et al. 2014. Fibroblast growth factor-21 is expressed in neonatal and pheochromocytoma-induced adult human brown adipose tissue. Metabolism: clinical and experimental 63:312-317), muscle (Izumiya, Y., et al. 2008. FGF21 is an Akt-regulated myokine. FEBS letters 582:3805-3810), and the endocrine and exocrine pancreas (Fon Tacer, K., et al. 2010. Research resource: Comprehensive expression atlas of the fibroblast growth factor system in adult mouse. Molecular endocrinology 24:2050-2064; Johnson, C. L., et al. 2009. Fibroblast growth factor 21 reduces the severity of cerulein-induced pancreatitis in mice. Gastroenterology 137:1795-1804; Omar, B. A., et al. 2014. Fibroblast growth factor 21 (FGF21) and glucagon-like peptide 1 contribute to diabetes resistance in glucagon receptor-deficient mice. Diabetes 63:101-110.120, 121). An elegant study by Markan et al. employed a liver specific FGF21 knockout mouse model (FGF21f1/f1;Albumin-Cre) to show the liver is the primary source of circulating FGF21 in vivo (Markan, K. R., et al. 2014. Circulating FGF21 is liver derived and enhances glucose uptake during refeeding and overfeeding. Diabetes 63:4057-4063). Both fasting and refeeding studies in these FGF21 liver-specific knock out mice showed circulating FGF21 levels were abolished, despite FGF21 mRNA expression being present in other expected tissues.

Commensurate with the numerous tissues which express FGF21, many mechanisms have been described with regard to regulation of its expression which contributes to the complexity of FGF21 biology. The diet is the most common known regulator of FGF21. In circulation, FGF21 has been shown to be elevated in conditions of fasting (Galman, C., et al. 2008. The circulating metabolic regulator FGF21 is induced by prolonged fasting and PPARalpha activation in man. Cell metabolism 8:169-174; Markan, K. R., et al. 2014. Circulating FGF21 is liver derived and enhances glucose uptake during refeeding and overfeeding. Diabetes 63:4057-4063; Muise, E. S., et al. 2008. Adipose fibroblast growth factor 21 is up-regulated by peroxisome proliferator-activated receptor gamma and altered metabolic states. Molecular pharmacology 74:403-412), ketogenic diets (Muise, E. S., et al. 2008. Adipose fibroblast growth factor 21 is up-regulated by peroxisome proliferator-activated receptor gamma and altered metabolic states. Molecular pharmacology 74:403-412), amino acid deprivation or low protein diets (De Sousa-Coelho, A. L., et al. 2012. Activating transcription factor 4-dependent induction of FGF21 during amino acid deprivation. The Biochemical journal 443:165-171; De Sousa-Coelho, A. L., et al. 2013. FGF21 mediates the lipid metabolism response to amino acid starvation. Journal of lipid research 54:1786-1797; Laeger, T., et al. 2014. FGF21 is an endocrine signal of protein restriction. The Journal of clinical investigation 124:3913-3922; Lees, E. K., et al. 2014. Methionine restriction restores a younger metabolic phenotype in adult mice with alterations in fibroblast growth factor 21. Aging cell 13:817-827; Ozaki, Y., et al. 2015. Rapid increase in fibroblast growth factor 21 in protein malnutrition and its impact on growth and lipid metabolism. The British journal of nutrition 114:1410-1418; Gosby, A. K., et al. 2016. Raised FGF21 and Triglycerides Accompany Increased Energy Intake Driven by Protein Leverage in Lean, Healthy Individuals: A Randomised Trial. PloS one 11:e0161003; Solon-Biet, S. M., et al. 2016. Defining the Nutritional and Metabolic Context of FGF21 Using the Geometric Framework. Cell metabolism 24:555-565), obesity and diabetes (Hale, C., et al. 2012. Lack of overt FGF21 resistance in two mouse models of obesity and insulin resistance. Endocrinology 153:69-80), and cold-exposure (Hondares, E., et al. 2011. Thermogenic activation induces FGF21 expression and release in brown adipose tissue. The Journal of biological chemistry 286:12983-12990). As PPARα can be elevated in many of the abovementioned states (fasting, ketogenic diets, obesity), PPARα agonist administration has also been shown to increase circulating FGF21 (Galman, C., et al. 2008. The circulating metabolic regulator FGF21 is induced by prolonged fasting and PPARalpha activation in man. Cell metabolism 8:169-174, 72; Muise, E. S., et al. 2008. Adipose fibroblast growth factor 21 is up-regulated by peroxisome proliferator-activated receptor gamma and altered metabolic states. Molecular pharmacology 74:403-412; Christodoulides, C. et al. 2009. Circulating fibroblast growth factor 21 is induced by peroxisome proliferator-activated receptor agonists but not ketosis in man. The Journal of clinical endocrinology and metabolism 94:3594-3601). Current tissue specific regulatory mechanisms are considered below.

FGF21 requires a co-factor, β-Klotho, to securely bind and activate FGFR (Ogawa, Y., et al. 2007. BetaKlotho is required for metabolic activity of fibroblast growth factor 21. Proceedings of the National Academy of Sciences of the United States of America 104:7432-7437). Micanovic et al. confirmed the C terminus is responsible for the interaction between FGF21 and β-Klotho (Micanovic, R., et al. 2009. Different roles of N- and C- termini in the functional activity of FGF21. Journal of cellular physiology 219:227-234). This co-factor necessity confers signaling specificity of FGF21; only tissues that express β-Klotho respond to FGF21. β-Klotho expression confers the responsiveness of a given tissue to FGF21, and tissues known to express β-Klotho are the liver (Fon Tacer, K., et al. 2010. Research resource: Comprehensive expression atlas of the fibroblast growth factor system in adult mouse. Molecular endocrinology 24:2050-2064), white and brown adipose tissues (Fisher, F. M., et al. 2011. Integrated regulation of hepatic metabolism by fibroblast growth factor 21 (FGF21) in vivo. Endocrinology 152:2996-3004; Ogawa, Y., et al. 2007. BetaKlotho is required for metabolic activity of fibroblast growth factor 21. Proceedings of the National Academy of Sciences of the United States of America 104:7432-7437, 147; Fon Tacer, K., et al. 2010. Research resource: Comprehensive expression atlas of the fibroblast growth factor system in adult mouse. Molecular endocrinology 24:2050-2064, Arnr, P., et al. 2008. FGF21 attenuates lipolysis in human adipocytes—a possible link to improved insulin sensitivity. FEBS letters 582:1725-1730), hypothalamus (Bookout, A. L., et al. 2013. FGF21 regulates metabolism and circadian behavior by acting on the nervous system. Nature medicine 19:1147-1152; Liang, Q., et al. 2014. FGF21 maintains glucose homeostasis by mediating the cross talk between liver and brain during prolonged fasting. Diabetes 63:4064-4075), and the exocrine and endocrine pancreas (Fon Tacer, K., et al. 2010. Research resource: Comprehensive expression atlas of the fibroblast growth factor system in adult mouse. Molecular endocrinology 24:2050-2064).

FGF21 receptor and co-receptor, β-Klotho, are expressed in both mouse and human placenta. β-Klotho expression is decreased in states of chronically elevated FGF21 in liver and white adipose tissue as shown in ob/ob mice and DIO mice. Further, rescuing β-Klotho expression restores FGF21 action.

```
Homo sapiens klotho beta (KLB), mRNA (NCBI Reference Sequence
NM_175737; SEQ ID NO: 1)
      1 atcctcagtc tcccagttca agctaatcat tgacagagct ttacaatcac aagcttttac
    61 tgaagctttg ataagacagt ccagcagttg gtggcaaatg aagccaggct gtgcggcagg
  121 atctccaggg aatgaatgga ttttcttcag cactgatgaa ataaccacac gctataggaa
  181 tacaatgtcc aacgggggat tgcaaagatc tgtcatcctg tcagcactta ttctgctacg
  241 agctgttact ggattctctg gagatggaag agctatatgg tctaaaaatc ctaattttac
  301 tccggtaaat gaaagtcagc tgtttctcta tgacactttc cctaaaaact ttttctgggg
  361 tattgggact ggagcattgc aagtggaagg gagttggaag aaggatggaa aaggaccttc
  421 tatatgggat catttcatcc acacacacct taaaaatgtc agcagcacga atggttccag
  481 tgacagttat atttttctgg aaaaagactt atcagccctg gattttatag gagtttcttt
  541 ttatcaattt tcaatttcct ggccaaggct tttccccgat ggaatagtaa cagttgccaa
  601 cgcaaaaggt ctgcagtact acagtactct tctggacgct ctagtgctta gaaacattga
  661 acctatagtt actttatacc actgggattt gcctttggca ctacaagaaa aatatggggg
  721 gtggaaaaat gataccataa tagatatctt caatgactat gccacatact gtttccagat
  781 gtttggggac cgtgtcaaat attggattac aattcacaac ccatatctag tggcttggca
  841 tgggtatggg acaggtatgc atgcccctgg agagaaggga aatttagcag ctgtctacac
  901 tgtgggacac aacttgatca aggctcactc gaaagtttgg cataactaca acacacattt
  961 ccgcccacat cagaagggtt ggttatcgat cacgttggga tctcattgga tcgagccaaa
1021 ccggtcggaa aacacgatgg atatattcaa atgtcaacaa tccatggttt ctgtgcttgg
1081 atggtttgcc aaccctatcc atggggatgg cgactatcca gaggggatga gaaagaagtt
1141 gttctccgtt ctacccattt tctctgaagc agagaagcat gagatgagag gcacagctga
1201 tttctttgcc ttttcttttg gacccaacaa cttcaagccc ctaaacacca tggctaaaat
1261 gggacaaaat gtttcactta atttaagaga agcgctgaac tggattaaac tggaatacaa
1321 caaccctcga atcttgattg ctgagaatgg ctggttcaca gacagtcgtg tgaaaacaga
1381 agacaccacg gccatctaca tgatgaagaa tttcctcagc caggtgcttc aagcaataag
1441 gttagatgaa atacgagtgt ttggttatac tgcctggtct ctcctggatg gctttgaatg
1501 gcaggatgct tacaccatcc gccgaggatt attttatgtg gattttaaca gtaaacagaa
1561 agagcggaaa cctaagtctt cagcacacta ctacaaacag atcatacgag aaaatggttt
1621 ttctttaaaa gagtccacgc cagatgtgca gggccagttt ccctgtgact tctcctgggg
1681 tgtcactgaa tctgttctta agcccgagtc tgtggcttcg tccccacagt tcagcgatcc
1741 tcatctgtac gtgtggaacg ccactggcaa cagactgttg caccgagtgg aaggggtgag
1801 gctgaaaaca cgacccgctc aatgcacaga ttttgtaaac atcaaaaaac aacttgagat
1861 gttggcaaga atgaaagtca cccactaccg gtttgctctg gattgggcct cggtccttcc
1921 cactggcaac ctgtccgcgg tgaaccgaca ggccctgagg tactacaggt gcgtggtcag
1981 tgaggggctg aagcttggca tctccgcgat ggtcaccctg tattatccga cccacgccca
2041 cctaggcctc cccgagcctc tgttgcatgc cgacgggtgg ctgaacccat cgacggccga
2101 ggccttccag gcctacgctg ggctgtgctt ccaggagctg ggggacctgg tgaagctctg
2161 gatcaccatc aacgagccta accggctaag tgacatctac aaccgctctg gcaacgacac
2221 ctacggggcg gcgcacaacc tgctggtggc ccacgccctg gcctggcgcc tctacgaccg
2281 gcagttcagg ccctcacagc gcggggccgt gtcgctgtcg ctgcacgcgg actgggcgga
2341 acccgccaac ccctatgctg actcgcactg gagggcggcc gagcgcttcc tgcagttcga
2401 gatcgcctgg ttcgccgagc cgctcttcaa gaccggggac taccccgcgg ccatgaggga
2461 atacattgcc tccaagcacc gacgggggct ttccagctcg gccctgccgc gcctcaccga
2521 ggccgaaagg aggctgctca agggcacggt cgacttctgc gcgctcaacc acttcaccac
2581 taggttcgtg atgcacgagc agctggccgg cagccgctac gactcggaca gggacatcca
2641 gtttctgcag gacatcaccc gcctgagctc ccccacgcgc ctggctgtga ttccctgggg
2701 ggtgcgcaag ctgctgcggt gggtccggag gaactacggc gacatggaca tttacatcac
2761 cgccagtggc atcgacgacc aggctctgga ggatgaccgg ctccggaagt actacctagg
2821 gaagtacctt caggaggtgc tgaaagcata cctgattgat aaagtcagaa tcaaaggcta
2881 ttatgcattc aaactggctg aagagaaatc taaacccaga tttggattct tcacatctga
2941 ttttaaagct aaatcctcaa tacaatttta caacaaagtg atcagcagca ggggcttccc
3001 ttttgagaac agtagttcta gatgcagtca gacccaagaa aatacagagt gcactgtctg
3061 cttattcctt gtgcagaaga aaccactgat attcctgggt tgttgcttct tctccaccct
3121 ggttctactc ttatcaattg ccatttttca aaggcagaag agaagaaagt tttggaaagc
3181 aaaaaactta caacacatac cattaaagaa aggcaagaga gttgttagct aaactgatct
3241 gtctgcatga tagacagttt aaaaattcat cccagttcca tatgctggta acttacagga
3301 gatatacctg tattatagaa agacaatctg agatacagct gtaaccaagg tgatgacaat
3361 tgtctctgct gtgtggttca aagaacattc ccttaggtgt tgacatcagt gaactcagtt
3421 cttggatgta aacataaagg cttcatcctg acagtaagct atgaggatta catgctacat
3481 tgcttcttaa agtttcatca actgtattcc atcattctgc tttagctttc atctctacca
3541 atagctactt gtggtacaat aaattatttt taagaagtaa aactctgggg ctggacgctg
3601 tggctcacac ctgtaatctc agcactttgg gaggccgagg cggggagatc acctgaggtg
3661 aggagttcga gaccagcctg gccaacatgg tgaaaccatg tctctactaa aaatacaaaa
3721 aattagccag gcgtggtgac agtggcacct gtaatcccag ctacttggga ggctgaggca
3781 gaagtttgaa cccaggaaac aggttacagt aggccaaaat tgcgccactg cactccagcc
3841 taggcgacaa cagcaagact gtgtccaaaa aaaaaaaaaa aagcaaaagc aaaactttgt
3901 tttgttagac tctacagcag agatttaaca cccttcttta aactgggtag tcagtgatag
3961 ataatatata ttctgtcact tctaataagg tgccttctcc tttaggtcag ggtggttcta
4021 aaatggaaag aaaacacaat agggtaagta gtgcttgtct aagccagtta caacacagac
4081 tcttaaagag gatcaagccc ttcatttttc taacaacaaa aaatcaccta tagaatatct
4141 aatttgtgat cttttactag atctgatttt ttaaaataat gtaatttccg gccaggcacg
4201 gtggcaccgc ctgtaatccc agcactttgg gaggccaagg caggtggatc acctgaggtt
4261 aggagttcga gactagcctg gccaacatgg caaaacccca tctctactaa aaatacaaaa
4321 gttagccggg catggtggtg ggcacctgta atcccagcta ctcaggaggc cgaggcagga
4381 gaatcgcttg aacccgagag gcagaggttg caatgagcca agatcgtgcc attgcactcc
4441 agcctggggg acagggcaag actgtctctc aaaataaaaa aaaataataa aaataaaaat
4501 aaaagtaatt tccaaaacct catctcatgg aaagatcaca ggatgaagga aagctagact
4561 caactctgtg aatagaagtt gctatactgt aagtaaagca acaattcaga atactgaatg
4621 agtttaaatt gttttatata gcaccctttt gggctagggt taattactag atctgacttg
4681 gataatttga cactttggga aatgaactct gttcttgaga cttgttcagt gtattttaaa
4741 catctgagga agaaaactta aatatgcacc tatttatacc tattctttct ttaggtcaac
4801 atttaacacc cactgcatac attaatttgt ccttgtctgc tcactccagc aatttagacc
4861 ttaacagtca caagagacgt tcttctgtta caaagcctta gtaaattaag gcagttttga
4921 ttatattcta ggtccaccta tgtctgaagc taaattcagt atctaactgc taatgaacaa
4981 gtttccaaaa tactgtaaaa atacaattag tcaatttgag taaatgcaaa tatgatgaga
5041 aatcaatttg ctatttggcc tggcaaatgg gaacagtaaa attctgcttt actcttctct
5101 agtctccttg ccccagctgc acccactacc ccaaagttgg cagttttgag gtatgatttt
5161 caaggaattt ttttagtatt aacatctccc tctgagaact atgtacctaa ggtcacgcat
5221 acaactagtc aattctgttt ttattactct aactatgtag aaacagtaag tcacttaaaa
5281 caatcacttg gctgggtttt ttcccctttg tgccacattg attcaccctg acccaagaac
5341 tccagggaaa attctttaat gtcaactggg caactcatta acctctcttt aacatcaagg
5401 gcttgggaaa aaaaaaaaaa aggttagcca caggaataac aaaaacctgg aatttatctt
5461 tcaggttttg ctttctcttt ctcactttgt ttaaagtatc tcgtactcac agttcacaaa
5521 ttaaccttca ctgtctcttt cacattaaga gcttatgctt aaagcatgcc ccccttttct
5581 aacttgctgg tttaccataa actcccctaa gtaataaaat tcctaaccca gtactgagag
5641 tcctccttct ctgccacttg ggcattattt tactagtttt taagccatca tcgcacaaga
5701 atccaaaaac ccttaaattt tttaaccact ggcaaatatg tacagcaaat taggttaagc
5761 atttaatctg gctcatgctc tatcatacta aatattcagg tttatcataa actccttaaa
5821 aaccatcaaa ggtcaaccag aaactgataa ctcttgaaag gagcaaacag gtaagatctt
5881 tggagtttaa gcttttctga gatgtgttgt gaaaaatcta acgtgtttat cgtatattca
5941 atgtaacaac ctggagaatc acaactatat ttaaagagcc tctggaaaat gaggccagta
6001 cagtgtgact acatgtttaa ttttcaatgt aatttattcc aaataaactg gttcatgctg
6061 accacttgta ttcaactaa
Homo sapiens beta-klotho, protein (NCBI Reference Sequence: NP_783864;
SEQ ID NO: 2)
      1 mkpgcaagsp gnewiffstd eittryrntm sngglqrsvi lsalillrav tgfsgdgrai
    61 wsknpnftpv nesqlflydt fpknffwgig tgalqvegsw kkdgkgpsiw dhfihthlkn
  121 vsstngssds yiflekdlsa ldfigvsfyq fsiswprlfp dgivtvanak glqyystlld
  181 alvlrniepi vtlyhwdlpl alqekyggwk ndtiidifnd yatycfqmfg drvkywitih
  241 npylvawhgy gtgmhapgek gnlaavytvg hnlikahskv whnynthfrp hqkgwlsitl
  301 gshwiepnrs entmdifkcq qsmvsvlgwf anpihgdgdy pegmrkklfs vlpifseaek
  361 hemrgtadff afsfgpnnfk pintmakmgq nvslnlreal nwikleynnp riliaengwf
  421 tdsrvktedt taiymmknfl sqvlqairld eirvfgytaw slldgfewqd aytirrglfy
  481 vdfnskqker kpkssahyyk qiirengfsl kestpdvqgq fpcdfswgvt esvlkpesva
  541 sspqfsdphl yvwnatgnrl lhrvegvrlk trpaqctdfv nikkqlemla rmkvthyrfa
  601 ldwasvlptg nlsavnrgal ryyrcvvseg lklgisamvt lyypthahlg 1pepllhadg
  661 wlnpstaeaf qayaglcfge lgdlvklwit inepnrlsdi ynrsgndtyg aahnllvaha
  721 lawrlydrqf rpsqrgaysl slhadwaepa npyadshwra aerflqfeia wfaeplfktg
  781 dypaamreyi askhrrglss salprlteae rrllkgtvdf calnhfttrf vmheqlagsr
  841 ydsdrdiqfl qditrlsspt rlavipwgvr kllrwvrrny gdmdiyitas giddqaledd
  901 rlrkyylgky lqevlkayli dkvrikgyya fklaeekskp rfgfftsdfk akssiqfynk
  961 vissrgfpfe nsssrcsqtq entectvclf lvqkkplifl gccffstivl llsiaifqrq
1021 krrkfwkakn lqhiplkkgk rvvs
```

Fetal Growth Restriction

Intrauterine Growth Restriction (IUGR), also known as Fetal Growth Restriction, is when a fetus in the womb fails to grow at the expected rate during the pregnancy. In other words, at any point in the pregnancy, the baby is not as big as would be expected for how far along the mother is in her pregnancy (this timing is referred to as an unborn baby's “gestational age”).

“Fetus” can refer to an unborn child when it is still in the mother's uterus.

“Neonatal”, or “neonate” and “newborn” can be used interchangeably and can refer to a newborn child and the time period directly after birth.

“Infant” and “baby” can be used interchangeably and refer to a newborn baby and its first year of life.

“Preterm” can refer to a birth that takes place before week 37 of pregnancy.

Babies who have fetal growth restriction often have a low weight at birth. If the weight is below the 10th percentile for a baby's gestational age (meaning that 90% of babies that age weigh more) the baby is also referred to as “small for gestational age,” or SGA.

IUGR can be classified as symmetrical IUGR, in which a baby's body is proportionally small (meaning all parts of the baby's body are similarly small in size), or asymmetrical IUGR, which is when the baby has a normal-size head and brain but the rest of the body is small. Infant head circumference is a reliable clinical indicator for nutritional insufficiency in utero. 60-70% of adult head circumference is achieved by the time a fetus is born, and 80-90% of adult head circumference is achieved by the age of 2.

Fundal height, i.e. measurement of the size of the uterus across a patient's abdomen with a tape measure, is currently the only routine method employed in the clinical setting for detection of fetal growth restriction or retardation (of note, ultrasound imaging which may be implemented if a patient has a higher risk or predisposition to an IUGR fetus). When considering the potential consequences of growth restriction for a fetus, including fetal mortality, the needed for more reliable assessments for growth restriction to supplement this archaic and highly unreliable assessment is apparent.

Currently, there is no blood test for identification and/or diagnosis of fetal growth restriction during pregnancy. Measuring circulating FGF21 during pregnancy has the potential to improve the accuracy of detection of growth restriction, as well as minimize the risk for growth restriction to be undetected.

Aspects of the invention are directed towards a non-invasive method of identifying a fetus at risk for or diagnosing a fetus with fetal growth restriction. In embodiments, the method comprises measuring the level of FGF21 protein or fragments thereof in a biological sample obtained from a subject pregnant with the fetus, and identifying the fetus as at risk for fetal growth restriction when FGF21 protein levels in the sample are elevated above control levels. Such fragments are described herein, such as N-terminal truncated FGF21. For example, methods of measuring the level of FGF21 protein levels in a sample obtained from a pregnant mother can be found in Sutton, Elizabeth F., et al. “Fibroblast growth factor 21, adiposity, and macronutrient balance in a healthy, pregnant population with overweight and obesity.” Endocrine research (2018): 1-9, which is incorporated by reference herein in its entirety.

As used herein, the term “FGF21” can refer to a member of the fibroblast growth factor (FGF) protein family. For example, an amino acid sequence of FGF21 (GenBank Accession No. NP 061986.1) is set forth as SEQ ID NO: 3, the corresponding polynucleotide sequence of which is set forth as SEQ ID NO: 4 (NCBI reference sequence number NM 019113.2). “FGF21” can also refer to the mature, intact polypeptide corresponding to SEQ ID NO. 6, which lacks the signal peptide (SEQ ID NO. 8).

As used herein, the term “FGF21 receptor” can refer to a receptor for FGF21 (Kharitonenkov, A, et al. (2008) Journal of Cellular Physiology 215: 1-7; Kurosu, H, et al. (2007) JBC 282:26687-26695; Ogawa, Y, et al. (2007) PNAS 104:7432-7437).

In embodiments, the method comprises measuring the level of FGF21 protein or fragments thereof in a biological sample obtained from a subject pregnant with the fetus, and identifying the fetus as at risk for fetal growth restriction when FGF21 protein levels in the sample are elevated above a threshold. The term “threshold”, for example an FGF21 threshold, refers to a value derived from a plurality of biological samples, such as donor blood samples, for a biomarker, such as a polypeptide selected from SEQ ID NO:3, above which threshold is associated with an increased likelihood of having and/or developing fetal growth restriction. For example the threshold or “threshold value” can be obtained by a Receiver Operating Characteristic (ROC) analysis on a large same with several hundred cases of confirmed fetal growth restriction.

In embodiments, the subject sample can be compared to a one or more control samples as described herein. For example, the amount of FGF21 in a biological sample may be compared to that of a control sample, such as a sample from a normal, untreated, or abnormal state control sample, so as to identify a subject with fetal growth restriction or a subject at risk of having fetal growth restriction. Determination of statistical significance is within the ability of those skilled in the art, e.g., the number of standard deviations from the mean that constitute a positive or negative result.

The term “subject” or “patient” can refer to any organism to which aspects of the invention can be administered, e.g., for experimental, diagnostic, prophylactic, and/or therapeutic purposes. Typical subjects to which compounds of the present disclosure may be administered will be mammals, particularly primates, especially humans. An exemplary subject, for example, comprises a pregnant human. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive. The term “living subject” refers to the entire subject or organism and not just a part excised (e.g., a liver or other organ) from the living subject.

In embodiments, FGF21 or fragments thereof, such as SEQ ID NO. 5, SEQ ID NO. 6, or SEQ ID NO. 7, can be measured or detected during any stage of pregnancy, such as during the 2nd trimester and/or during the 3rd trimester. During these later stages of gestation, the demand for protein to provide and laydown fetal material is dramatically increased. The term “pregnancy” refers to the nine months (40 weeks from the last menstrual period) of pregnancy which is traditionally divided into three trimesters, i.e., distinct periods of roughly three months in which different phases of fetal development take place. The first trimester is a time of basic cell differentiation. It is believed to end at the mother's first perception of fetal movement (quickening), which usually occurs around the end of the third month (or about 12 to about 14 weeks of gestational age). The second trimester is a period of rapid growth and maturation of body systems (about 15 to about 28 weeks of gestational age). A second-trimester fetus born prematurely may be viable, depending on the hospital care. The third trimester marks the final stage of fetal growth, in which systems are completed, fat accumulates under the fetus' skin, and the fetus moves into position for birth (about 29 to about 42 weeks of gestational age). This trimester ends with the birth itself.

The fibroblast growth factor 21 precursor (Homo sapiens) is a protein comprising 209 amino acids (NCBI reference sequence number NP_061986) as set forth in SEQ ID NO: 3, the corresponding polynucleotide sequence of which is set forth as SEQ ID NO: 4 (NCBI reference sequence number NM_019113). Amino acid residues 1-28 of SEQ ID NO: 3 correspond to the signal peptide (SEQ ID NO: 8), whereas amino acid residues 29-209 correspond to intact FGF21 (SEQ ID NO: 6), also refered to as mature FGF21.

The physiological functions of FGF21 rely on the intact molecular structure of FGF21, and amino acid sequence in its N-terminal and C-terminal region. Fragments of peptides of certain fibroblast growth factors are biologically active. See for example, Baird et al, Proc. Natl. Acad. Sci (USA) 85:2324-2328 (1988), and J. Cell. Phys. Suppl. 5: 101-106 (1987). For example, N-terminal truncated FGF21 (7-181) (SEQ ID NO: 5) is a potent inhibitor that competitively inhibits the biological activity of intact, mature FGF21 (1-181) (SEQ ID NO: 6). Further, the N-terminus of FGF21 (HisProIlePro) contains two dipeptides that could potentially be substrates to dipeptidyl peptidase IV (DPP-IV), a serine type protease involved in inactivation of neuropeptides, endocrine peptides, and cytokines (Damme et al. Chem. Immunol. 72: 42-56, (1999)), resulting in a fragment of FGF21 truncated at the N-terminus by 4 amino acids (SEQ ID NO: 7). Embodiments of the invention comprise measuring intact FGF21 level, such as circulating intact FGF21, or fragments thereof, such as N-terminal truncated FGF21 or C-terminal truncated FGF21, in the assessment of fetal growth restriction. Thus, the term “FGF21”, “FGF21 protein” or “FGF21 polypeptide” can refer to a FGF21 polypeptide expressed in humans. For purposes of this disclosure, the term “FGF21 polypeptide” or “FGF21 protein” can be used interchangeably to refer to any full-length FGF21 polypeptide, e.g., SEQ ID NO: 3, which consists of 209 amino acid residues; any mature form of the polypeptide, such as the polypeptide consisting of 181 amino acid residues, and in which the 28 amino acid residues at the amino-terminal end of the full-length FGF21 polypeptide (i.e., which constitutes the signal peptide) have been removed; or variants or truncated forms thereof. In one embodiment, a fragment of SEQ ID NO:3 or SEQ ID NO: 6 can be at least 3, at least 5, at least 7, at least 10, at least 20, at least 30, at least 40, at least 50, at least 60, at least 70, at least 80, at least 90. at least 100, at least 110, at least 120, at least 130, at least 140, at least 150, at least 160, or at least 170 amino acids in length. In one embodiment, a truncated form of the mature FGF21 polypeptide comprising SEQ ID NO: 3 can be at least 100, at least 110, at least 120, at least 130, at least 140, at least 150, at least 160, at least 170, or at least 180 consecutive amino acids in length. In some embodiments, the truncation can be an N-terminal truncation or a C-terminal truncation. In one embodiment, a variant of the mature FGF21 polypeptide can be at least 50%, 55%, 60%, 65%, 70%, 75%, 80%, 85%, 90%, 91%, 92%, 93%, 94%, 95%, 96%, 97%, 98%, or 99% identical to SEQ ID NO: 3.

Embodiments of the invention comprise measuring FGF21 or a fragment thereof. For example, embodiments comprise measuring FGF21 or fragment as set forth in SEQ ID NO: 3 (NCBI reference sequence number NP 061986), the corresponding polynucleotide sequence of which is set forth as SEQ ID NO: 4 (NCBI reference sequence number NM 019113).

```
Fibroblast growth factor 21 precursor - Homo sapiens (NCBI reference
sequence number NP 061986 (SEQ ID NO: 3), with intact FGF21 indicated
therein by bold and underline (see also SEQ ID NO: 6) and non-
italicized residues corresponding to the signal sequence (see also SEQ
ID NO: 8).
      1 mdsdetgfeh sglwvsvlag lllgacqa [symbol]
    61 [symbol] [symbol]
  121 [symbol] [symbol]
  181 [symbol] [symbol]
Homo sapien fibroblast growth factor 21 (NCBI reference sequence
number NM 019113; SEQ ID NO: 4)
      1 gaggcttcca aggcaggata cttgtgtctc agatgcggtc gcttctttca tacagcaatt
    61 gccgccttgc tgaggatcaa ggaacctcag tgtcagatca cgccctcccc ccaaacttag
  121 aaattcagat ggggcgcaga aatttctctt gttctgcgtg atctgcatag atggtccaag
  181 aggtggtttt tccaggagcc cagcacccct cctccctccg actcagaccc aggagtctgg
  241 ccctccattg aaaggacccc aggttacatc atccattcag gctgcccttg ccacgatgga
  301 attctgtagc tcctgccaaa tgggtcaaat atcatggttc aggcgcaggg agggtgattg
  361 ggcgggcctg tctgggtata aattctggag cttctgcatc tatcccaaaa aacaagggtg
  421 ttctgtcagc tgaggatcca gccgaaagag gagccaggca ctcaggccac ctgagtctac
  481 tcacctggac aactggaatc tggcaccaat tctaaaccac tcagcttctc cgagctcaca
  541 ccccggagat cacctgagga cccgagccat tgatggactc ggacgagacc gggttcgagc
  601 actcaggact gtgggtttct gtgctggctg gtcttctgct gggagcctgc caggcacacc
  661 ccatccctga ctccagtcct ctcctgcaat tcgggggcca agtccggcag cggtacctct
  721 acacagatga tgcccagcag acagaagccc acctggagat cagggaggat gggacggtgg
  781 ggggcgctgc tgaccagagc cccgaaagtc tcctgcagct gaaagccttg aagccgggag
  841 ttattcaaat cttgggagtc aagacatcca ggttcctgtg ccagcggcca gatggggccc
  901 tgtatggatc gctccacttt gaccctgagg cctgcagctt ccgggagctg cttcttgagg
  961 acggatacaa tgtttaccag tccgaagccc acggcctccc gctgcacctg ccagggaaca
  1021 agtccccaca ccgggaccct gcaccccgag gaccagctcg cttcctgcca ctaccaggcc
  1081 tgccccccgc actcccggag ccacccggaa tcctggcccc ccagcccccc gatgtgggct
  1141 cctcggaccc tctgagcatg gtgggacctt cccagggccg aagccccagc tacgcttcct
  1201 gaagccagag gctgtttact atgacatctc ctctttattt attaggttat ttatcttatt
  1261 tattttttta tttttcttac ttgagataat aaagagttcc agaggaggat aaaaaaaaaa
  1321 aaaaaaaaaa aaa
```

Embodiments of the invention comprise measuring polypeptide fragments of FGF21, such as circulating FGF21 polypeptide fragments. In embodiments, the fragment length comprises 2-10 amino acids, 11-20 amino acids, 21-30 amino acids, 31-40 amino acids, 41-50 amino acids, 51-60 amino acids, 61-70 amino acids, 71-80 amino acids, 81-90 amino acids, 91-100 amino acids, 101-110 amino acids, 111-120 amino acids, 121-130 amino acids, 131-140 amino acids, 141-150 amino acids, 151-160 amino acids, 161-170 amino acids, 171-180 amino acids, 181-190 amino acids, 191-200 amino acids, 201-209 amino acids.

In embodiments, FGF21, such as circulating FGF21, is measured and/or detected in a biological sample obtained from the mother, such as the pregnant mother. The biological sample can be a biological fluid, i.e., a bodily fluid. The bodily fluid comprises peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood. In some embodiments, the biological sample comprises blood or a blood derivative, such as peripheral blood, sera, or plasma. In other embodiments, the biological sample can be a biological tissue, such as placental tissue.

For example, a sample can be prepared to enhance detectability of the biomarkers. For example, a sample from the subject can be fractionated. Any method that enriches for a biomarker polypeptide of interest can be used. Sample preparations, such as prefractionation protocols, are optional and may not be necessary to enhance detectability of biomarkers depending on the methods of detection used. For example, sample preparation may be unnecessary if an antibody that specifically binds a biomarker is used to detect the presence of the biomarker in a sample. Sample preparation may involve fractionation of a sample and collection of fractions determined to contain the biomarkers. Methods of prefractionation include, for example, size exclusion chromatography, ion exchange chromatography, heparin chromatography, affinity chromatography, sequential extraction, gel electrophoresis and liquid chromatography.

The biological sample can serve as a test sample for an assay to detect and/or measure levels of a specific protein, such as FGF21 or β-Klotho. The results of the assay of the test sample is often indicative of the disease status of the subject. For example, in some cases, results of the assay of the test sample is indicative of the presence of the condition or disease in the subject, such as fetal growth restriction and/or nutrient insufficiency. In some cases, the assay involves incubating a biological fluid or fraction thereof with a protein binding agent, such as an antibody, so as to form an protein-binding agent complex. The assay further involves detecting and/or measuring the amount of protein-binding agent complex, for example by comparing the test sample to an appropriate standard or control sample. The results of the comparison can be indicative of whether the subject has the condition or disease. Thus, the subject may be diagnosed as having the condition or disease based on the results of the assay, in some cases.

In many cases, fetal growth restriction is the result of a problem that prevents a baby from getting enough oxygen and nutrients. This can result from a number of different reasons. For example, a common cause is placental insufficiency, in which the tissue that delivers oxygen and nutrients to the baby is not attached properly or isn't working correctly. Other possible causes during a woman's pregnancy include certain behaviors (such as smoking, drinking alcohol, or abusing drugs), exposure to infections passed from the mother (such as cytomegalovirus, German measles (rubella), toxoplasmosis, and syphilis), taking certain medications, high blood pressure, genetic disorders or birth defects, living in high altitudes and over-nutrition or under-nutrition or nutrient (eg. protein) insufficiency. Regardless of the underlying cause, this lack of nourishment slows the baby's growth, and can result in fetal growth restriction. There is a need to identify a subject at risk for fetal growth insufficiency.

Thus, aspects of the invention are also directed towards a non-invasive method of identifying a fetus exposed to nutrient insufficiency. In embodiments, the method comprises measuring the level of FGF21 in the sample obtained from a subject pregnant with the fetus, wherein a level of FGF21 in the sample elevated above control levels identifies a fetus exposed to nutrient insufficiency.

In embodiment, the biological same is obtained from a subject pregnant with a fetus, and the biological sample or fraction thereof is incubated with an protein binding agent as described herein, such as an FGF21 binding agent, so as to form an FGF21-binding agent complex. By contacting the biological sample with a protein binding agent, specific proteins or fragments thereof can be detected and/or measured.

In embodiments, the binding agent is an antibody or fragment thereof, such as an antibody specific for FGF21 (anti-FGF21); for example, the antibody binds specifically to an amino acid sequence fragment comprising SEQ ID NO: 3 and/or recognizes an epitope comprising a fragment of SEQ ID NO: 3. In some embodiments, the epitope comprising a fragment of SEQ ID NO: 3 can be can be at least 3, 4, 5, 6, 7, 8, 9, 10, 15, 20, 25, 30 or at least 50 amino acids in length. The antibody can be a polyclonal antibody or a monoclonal antibody. The antibody or fragment thereof can be attached to a molecule which is capable of identification, visualization, or localization using known methods. Suitable detectable labels include radioisotopic labels, enzyme labels, non-radioactive isotopic labels, fluorescent labels, toxin labels, affinity labels, and chemiluminescent labels. By detecting and/or measuring the level of FGF21 in a biological sample, for example, a clinician is able to determine one's risk for fetal growth restriction and/or nutrient insufficiency, and/or diagnose a fetus with fetal growth restriction and/or nutrient insufficiency. For example, FGF21 antibodies can be made according to well-established methods practiced by the skilled artisan or are commercially available to one of skill in the art (such as by suppliers antibodies-online (e.g., product no. ABIN1498256), Novus Biologicals (Littleton, Colo.; e.g., product no.: NBP2-00645), Abnova (Taiwan; e.g., product no.: PAB7549)).

The methods described herein involve obtaining a biological sample from the subject, such as a subject pregnant with a fetus. As used herein, the phrase “obtaining a biological sample” refers to any process for directly or indirectly acquiring a biological sample from a subject. For example, a biological sample may be obtained (e.g., at a point-of-care facility, e.g., a physician's office, a hospital, laboratory facility) by procuring a tissue or fluid sample (e.g., blood draw, marrow sample, spinal tap) from a subject. Alternatively, a biological sample may be obtained by receiving the biological sample (e.g., at a laboratory facility) from one or more persons who procured the sample directly from the subject. The biological sample may be, for example, a tissue (e.g., blood), cell (e.g., hematopoietic cell such as hematopoietic stem cell, leukocyte, or reticulocyte, stem cell, or plasma cell), vesicle, biomolecular aggregate or platelet from the subject.

The method of claim 15, further comprising determining the risk for fetal growth restriction by measuring FGF21 protein levels in a biological sample isolated from the subject.

Compositions to Treat or Prevent Fetal Growth Restriction

Aspects of the invention are also directed towards methods of treating and/or preventing fetal growth restriction. The term “treating” can refer to partially or completely alleviating, ameliorating, improving, relieving, delaying onset of, inhibiting progression of, reducing severity of, and/or reducing incidence of one or more symptoms, features, or clinical manifestations of a particular disease, disorder, and/or condition, such as fetal growth restriction. For example, “treating” fetal growth restriction can refer to increasing uptake of protein, such as in one's diet, or administering protein to a subject, such as with a pharmaceutical composition. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

“Treating fetal growth restriction,” for example, can mean increasing the size and/or weight of the fetus while in utero. Small size at birth has been demonstrated to adversely program a fetus for increased disease risk later in life (such as during adolescence and adulthood), such as increased risk for obesity, diabetes, hypertension, cardiovascular disease, etc.

There are also immediate risks to being born small—small babies have increased morbidity and mortality at delivery.

In embodiments, treating a subject comprises administering to a subject pregnant with a fetus determined to be afflicted with or at risk of fetal growth restriction an amount of protein sufficient to reduce circulating FGF21 levels and/or restore circulating FGF21 levels, such as to control levels.

The dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

A therapeutically effective dose can depend upon a number of factors known to those of ordinary skill in the art. The dose(s) can vary, for example, depending upon the identity, size, and condition of the subject or sample being treated, further depending upon the route by which the composition is to be administered, if applicable, and the effect which the practitioner desires. These amounts can be readily determined by the skilled artisan.

For example, the amount of protein sufficient to reduce or restore circulating FGF21, such as to control levels, comprises no less than about 0.88 grams of protein/kg of body weight/day. In some embodiments, the therapeutically effective amount is at least about 0.1 g/kg body weight, at least about 0.25 g/kg body weight, at least about 0.5 g/kg body weight, at least about 0.75 g/kg body weight, at least about 1 g/kg body weight, at least about 2 g/kg body weight, at least about 3 g/kg body weight, at least about 4 g/kg body weight, at least about 5 g/kg body weight, at least about 6 g/kg body weight, at least about 7 g/kg body weight, at least about 8 g/kg body weight, at least about 9 g/kg body weight, at least about 10 g/kg body weight, at least about 15 g/kg body weight, at least about 20 g/kg body weight, at least about 25 g/kg body weight, at least about 30 g/kg body weight, at least about 40 g/kg body weight, at least about 50 g/kg body weight, at least about 75 g/kg body weight, at least about 100 g/kg body weight, at least about 200 g/kg body weight, at least about 250 g/kg body weight, at least about 300 g/kg body weight, at least about 3500 g/kg body weight, at least about 400 g/kg body weight, at least about 450 g/kg body weight, at least about 500 g/kg body weight, at least about 550 g/kg body weight, at least about 600 g/kg body weight, at least about 650 g/kg body weight, at least about 700 g/kg body weight, at least about 750 g/kg body weight, at least about 800 g/kg body weight, at least about 900 g/kg body weight, or at least about 1000 g/kg body weight.

The protein can be administered to a subject in the form of a “food ingredient,” which refers to any edible substance that is combined is with other edible substances, where the final combination is consumed as a food. The term “medical food” herein is defined by statute in the United States of America, Orphan Drug Act, section 5(b) (21 U.S.C. 360ee (b) (3)), which defines “medical food” as “a food which is formulated to be consumed or administered enterally under the supervision of a physician and which is intended for the specific dietary management of a disease or condition for which distinctive nutritional requirements, based on recognized scientific principles, are established by medical evaluation.”

Other aspects of the invention are directed towards methods of treating fetal growth restriction by administering to a subject pregnant with a fetus afflicted with or at risk of fetal growth restriction a therapeutically effective amount of an agent that reduces the circulating protein level of FGF21. For example, the agent can be a synthetic polynucleotide, such as one that is targeted to the nucleic acid molecule encoding FGF21 as in NCBI reference sequence number NM_019113 (SEQ ID NO: 4), the nucleic acid molecule encoding β-klotho as in NCBI reference sequence NM_175737 (SEQ ID NO: 1), or a combination thereof. In embodiments, the sequence of the polynucleotide will be known to the skilled artisan. For example, FGF21 is knocked down in human HepG2 cells using siRNA oligonucleotide of sequence 5′-GCCUUGAAGCCGGGAGUUA-3′ (SEQ ID NO: 9) (see Kim, H. W., et al. Endocrinology 154.9 (2013): 3366-3376, which is incorporated by reference herein in its entirety). In another example, FGF21 is knocked down in human embryonic kidney (HEK) cells using multiple small hairpin RNA (shRNA) sequences, such as 5′-CCGGCTGAGCATGGTAGAGCCTTTACTCGAGTAAAGGCTCTACCATGCTCAGTTT TTG-3′ (SEQ ID NO: 10), among others. (see Leng, Yan, et al Molecular psychiatry 20.2 (2015): 215 and related supplemental information, the entireties of each of which are incorporated by reference herein). Further, see Badman, Michael K., et al. Cell metabolism 5.6 (2007): 426-437; Li, Ke, et al. Molecular and cellular endocrinology 348.1 (2012): 21-26; Wang, Cong, et al. The FEBS journal 281.9 (2014): 2136-2147. Jung, Jong Gab, et al. Molecules and cells 38.12 (2015): 1037; Leng, Yan, et al. International Journal of Neuropsychopharmacology 19.8 (2016): pyw035; Zhou, Yong, et al PloS one 11.7 (2016): e0159191; Loeffler, Ivonne, et al. Journal of the American Society of Nephrology 22.4 (2011): 649-663; Kim, H. W., et al. Endocrinology 154.9 (2013): 3366-3376; Wu, Shufang, et al. Journal of Biological Chemistry287.31 (2012): 26060-26067; Huang, Xiaohua, et al. Brain research 1530 (2013): 13-21; Lee, Jinmi, et al Metabolism63.8 (2014): 1041-1048; Wang, Ruiwei, et al. Biochemical and Biophysical Research Communications (2017); Yun, et al. Chinese medical journal 123.23 (2010): 3417-3421; and Wu, Haoshu, et al. Pharmacology 100.3-4 (2017): 115-126, the entireties of each of which are incorporated by reference herein.

The synthesis, compositions, and administration of such siRNA to a subject will be known to a skilled artisan (see for example, US20150038558A1, U.S. Pat. No. 7,022,828, and US20030190635, each of which are incorporated by reference in there entireties herein).

In embodiments, the agent is administered to the liver, white adipose tissue, brown adipose tissue, muscle, pancreas, or placenta. In exemplary embodiments, the agent is administered to the placenta of the subject. Without wishing to be bound by theory, administration to the placenta is of particular significance because it is through this organ that fetal growth restriction can be mediated. Without wishing to be bound by theory, the quantity of FGF21 peptide synthesized and secreted by the placenta is minimal and thus, placentally derived FGF21 can act by paracrine or autocrine manor through β-Klotho, specifically back on the placenta (i.e., acting on the tissue which produces it).

Agents and/or protein can be incorporated into pharmaceutical compositions suitable for administration. Such compositions can comprise the agent and a pharmaceutically acceptable carrier. Thus, in some embodiments, the agent or protein is present in a pharmaceutical composition.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Assays and Kits for Measuring Circulating FGF21

Aspects of the invention can comprise contacting the biological sample with a protein binding agent, such as an FGF21 binding agent or a β-klotho binding agent, to detect and/or measure specific proteins or fragments thereof in the sample. In embodiments, the binding agent is an anti-FGF21 binding agent.

Embodiments can measure or detect FGF21 or fragments thereof, such as N-terminal truncated FGF21, and/or can distinguish between intact FGF21 and truncated FGF21. For example, see FGF-21 (Intact) ELISA Assay Kit provided by Eagle, Bio (Nashua, NH).

Embodiments to measure, detect, distinguish, or differentiate such biomarkers can use assays known to the art. Non-limiting examples of assays include an immunoassay, a colorimetric assay, fluorimetric assay or a combination thereof. Non-limiting examples of immunoassays comprise a Western blot assay, an enzyme-linked immunosorbent assay (ELISA), immunoprecipitation or a combination thereof. For example, a biological sample collected from a subject can be incubated together with a biomarker specific antibody, such as an anti-FGF21 antibody or fragment thereof, and the binding of the antibody to the biomarker in the sample is detected or measured.

In embodiments, the antibody or fragment thereof may be specific for FGF21 (anti-FGF21). The antibody may be a polyclonal antibody or a monoclonal antibody. The antibody or fragment thereof may be attached to a molecule which is capable of identification, visualization, or localization using known methods. Suitable detectable labels include radioisotopic labels, enzyme labels, non-radioactive isotopic labels, fluorescent labels, toxin labels, affinity labels, and chemiluminescent labels.

Enzyme labels employed in embodiments herein, for example to detect protein levels or enzymatic activity, can be, for example, alkaline phosphatase, horseradish peroxidase, β-galactosidase and/or glucose oxidase; and the substrate can respectively be an alkaline phosphatase, horseradish peroxidase, β-galactosidase or glucose oxidase substrate (see Molecular Probes Handbook—A Guide to Fluorescent Probes and Labeling Technologies, 11th Edition (2010), Invitrogen, which is incorporated by reference herein in its entirety).

In embodiments, the enzyme, such as alkaline phosphatase or horseradish peroxidase, can be attached to a secondary antibody.

Alkaline phosphatase (AP) substrates include, but are not limited to, AP-Blue substrate (blue precipitate, Zymed catalog p. 61); AP-Orange substrate (orange, precipitate, Zymed), AP-Red substrate (red, red precipitate, Zymed), 5-bromo, 4-chloro, 3-indolyphosphate (BCIP substrate, turquoise precipitate), 5-bromo, 4-chloro, 3-indolyl phosphate/nitroblue tetrazolium/iodonitrotetrazolium (BCIP/INT substrate, yellow-brown precipitate, Biomeda), 5-bromo, 4-chloro, 3-indolyphosphate/nitroblue tetrazolium (BCIP/NBT substrate, blue/purple), 5-bromo, 4-chloro, 3-indolyl phosphate/nitroblue tetrazolium/iodonitrotetrazolium (BCIP/NBT/INT, brown precipitate, DAKO, Fast Red (Red), Magenta-phos (magenta), Naphthol AS-BI-phosphate (NABP)/Fast Red TR (Red), Naphthol AS-BI-phosphate (NABP)/New Fuchsin (Red), Naphthol AS-MX-phosphate (NAMP)/New Fuchsin (Red), New Fuchsin AP substrate (red), p-Nitrophenyl phosphate (PNPP, Yellow, water soluble), VECTOR™ Black (black), VECTOR™ Blue (blue), VECTOR™ Red (red), Vega Red (raspberry red color).

Horseradish Peroxidase (HRP, sometimes abbreviated PO) substrates include, but are not limited to, 2,2′ Azino-di-3-ethylbenz-thiazoline sulfonate (ABTS, green, water soluble), aminoethyl carbazole, 3-amino, 9-ethylcarbazole AEC (3A9EC, red). Alpha-naphthol pyronin (red), 4-chloro-1-naphthol (4C1N, blue, blue-black), 3,3′-diaminobenzidine tetrahydrochloride (DAB, brown), ortho-dianisidine (green), o-phenylene diamine (OPD, brown, water soluble), TACS Blue (blue), TACS Red (red), 3,3′,5,5′ Tetramethylbenzidine (TMB, green or green/blue), TRUE BLUE™ (blue), VECTOR™ VIP (purple), VECTOR™ SG (smoky blue-gray), and Zymed Blue HRP substrate (vivid blue).

Glucose Oxidase (GO) substrates, include, but are not limited to, nitroblue tetrazolium (NBT, purple precipitate), tetranitroblue tetrazolium (TNBT, black precipitate), 2-(4-iodophenyl)-5-(4-nitorphenyl)-3-phenyltetrazolium chloride (INT, red or orange precipitate), Tetrazolium blue (blue), Nitrotetrazolium violet (violet), and 3-(4,5-dimethylthiazol-2-yl)-2,5-diphenyltetrazolium bromide (MTT, purple). All tetrazolium substrates require glucose as a co-substrate. The glucose gets oxidized and the tetrazolium salt gets reduced and forms an insoluble formazan which forms the color precipitate.

Beta-Galactosidase substrates, include, but are not limited to, 5-bromo-4-chloro-3-indoyl beta-D-galactopyranoside (X-gal, blue precipitate).

Other examples of alkaline and acid phosphatase substrates comprise 9H-(1,3-dichloro-9,9-dimethylacridin-2-one-7-yl) phosphate, diammonium salt (DDAO phosphate), 6,8-difluoro-4-methylumbelliferyl phosphate (DiFMUP), fluorescein diphosphate, tetraammonium salt (FDP), 4-methylumbelliferyl phosphate, free acid (MUP), and 4-methylumbelliferyl phosphate, dicyclohexylammonium salt, trihydrate (MUP DCA salt).

Alkaline phosphatase activity, such as intestinal alkaline phosphatase activity, can be detected and/or measured with use of chromogenic substrates and/or fluorogenic substrates of alkaline phosphatases. For example, 4-methylumbelliferyl phosphate (MUP) is a fluorogenic substrate for alkaline phosphatases, and alkaline phosphatase mediated hydrolysis of its phosphate substituent yields the blue-fluorescent 4-methylumbelliferyl (excitation/emission 386/448 nm). In embodiments, the alkaline phosphatase substrate can be directly admixed with the biological sample, such as stool, allowing for the direct detection of the presence of alkaline phosphatase or the measurement of its activity.

Alkaline phosphatase (AP) substrates include, but are not limited to, AP-Blue substrate (blue precipitate, Zymed catalog p. 61); AP-Orange substrate (orange, precipitate, Zymed), AP-Red substrate (red, red precipitate, Zymed), 5-bromo, 4-chloro, 3-indolyphosphate (BCIP substrate, turquoise precipitate), 5-bromo, 4-chloro, 3-indolyl phosphate/nitroblue tetrazolium/ iodonitrotetrazolium (BCIP/INT substrate, yellow-brown precipitate, Biomeda), 5-bromo, 4-chloro, 3-indolyphosphate/nitroblue tetrazolium (BCIP/NBT substrate, blue/purple), 5-bromo, 4-chloro, 3-indolyl phosphate/nitroblue tetrazolium/iodonitrotetrazolium (BCIP/NBT/INT, brown precipitate, DAKO, Fast Red (Red), Magenta-phos (magenta), Naphthol AS-BI-phosphate (NABP)/Fast Red TR (Red), Naphthol AS-BI-phosphate (NABP)/New Fuchsin (Red), Naphthol AS-MX-phosphate (NAMP)/New Fuchsin (Red), New Fuchsin AP substrate (red), p-Nitrophenyl phosphate (PNPP, Yellow, water soluble), VECTOR™ Black (black), VECTOR™ Blue (blue), VECTOR™ Red (red), Vega Red (raspberry red color).

Other substrates known in the art, including those described herein, can be used with embodiments of the invention (see Molecular Probes Handbook—A Guide to Fluorescent Probes and Labeling Technologies, 11th Edition (2010), Invitrogen, which is incorporated by reference herein in its entirety). Further, various fluorophores known in the art can be covalently attached to the substrate, such as MUP.

Enzyme reactions can provide a highly specific, rapid and sensitive assay for detection of specific proteins in a sample, such as FGF21 and/or β-Klotho in stool. Examples of suitable fluorogenic substrates which can be utilized within the present invention comprise Fluorescein diacetate, 4-Methylumbelliferyl acetate, 4-Methylumbelliferyl casein, 4-Methylumbelliferyl-α-L-arabinopyranoside, 4-Methylumbelliferyl-β-D-fucopyranoside, 4-Methylumbelliferyl-α-L-fucopyranoside, 4-Methylumbelliferyl-β-L-fucopyranoside, 4-Methylumbelliferyl-α-D-galactopyranoside, 4-Methylumbelliferyl-β-D-galactopyranoside, 4-Methylumbelliferyl-α-D-glucopyranoside, 4-Methylumbelliferyl-β-D-glucopyranoside, 4-Methylumbelliferyl-β-D-glucuronide, 4-Methylumbelliferyl nonanoate, 4-Methylumbelliferyl oleate, 4-Methylumbelliferyl phosphate, bis(4-Methylumbelliferyl)phosphate, 4-Methylumbelliferyl pyrophosphate diester, 4-Methylumbelliferyl-β-D-xylopyranoside.

Non-limiting examples of suitable chromogenic substrates for use within the present invention comprise o-Nitrophenyl-β-D-galactopyranoside, p-Nitrophenyl-β-D-galactopyranoside, o-Nitrophenyl-β-D-glucopyranoside, p-Nitrophenyl-α-D-glucopyranoside, p-Nitrophenyl-β-D-glucopyranoside, p-Nitrophenyl-β-D-glucuronide, p-Nitrophenyl phosphate, o-Nitrophenyl-β-D-xylopyranoside, p-Nitrophenyl-α-D-xylopyranoside, p-Nitrophenyl-β-D-xylopyranoside, and Phenolphthalein-β-D-glucuronide.

Embodiments as described herein can be constructed so as to compare the level of FGF21 protein in the subject's sample to that of at least one control sample. As used herein, “changed as compared to a control” sample or subject is understood as having a level of the analyte or diagnostic or therapeutic indicator (e.g., marker), such as a peptide corresponding to FGF21 or a fragment thereon, to be detected at a level that is statistically different than a sample from a normal, untreated, or abnormal state control sample. Determination of statistical significance is within the ability of those skilled in the art, e.g., the number of standard deviations from the mean that constitute a positive or negative result.

Aspects of the invention are further directed towards a kit of molecular biomarkers for identifying a fetus at risk for growth restriction. In an embodiment, the kit comprises at least one bio-recognition element or binding agent for measuring levels of or detecting a protein, such as FGF21, and, optionally, at least a second bio-recognition element or binding agent for measuring levels of or detecting β-Klotho. The measurement or detection of two or more proteins, for example levels of FGF21 and β-Klotho elevated above control levels, together represent a molecular signature that is indicative of fetal growth restriction or nutrient insufficiency.

Additional components of kits of the invention may comprise a support structure and instructions for use thereof. For example, an FGF21 bio-recognition element, such as an antibody as described herein, may be immobilized to a solid support structure. Non-limiting examples of the composition of the solid support structure comprise plastic, cardboard, glass, plexiglass, tin, paper, or a combination thereof. The solid support may also comprise a dip stick, spoon, scoopula, filter paper or swab.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

FGF21 as a Diagnostic Tool to Identify Fetal Growth Restriction

Serum measurement of FGF21 levels in pregnant women for detection or diagnosis of restriction or retarded fetal growth, poor maternal nutrition status, or maternal/fetal nutrient insufficiencies.

Aspects of the invention are directed towards the use of the circulating hormone Fibroblast Growth Factor 21 (FGF21) as a biomarker during pregnancy for detection and/or diagnosis of fetal conditions, such as fetal growth restriction or growth retardation, failure to thrive syndrome, poor/insufficient maternal nutrition status, and/or maternal and/ or fetal nutrient insufficiency.

Aspects of the invention are directed towards compositions, methods and kits that can improve the detection of and/or treat/prevent the abovementioned conditions which, in turn, can minimize their adverse consequences such as maternal and fetal morbidity and mortality.

Fundal height, i.e. measurement of the size of the uterus across a patient's abdomen with a tape measure, is currently the only routine method employed in the clinical setting for detection of fetal growth restriction or retardation (of note, ultrasound imaging may be implemented if a patient has a higher risk or predisposition to an IUGR fetus). Moreover, there are no clinical assessments available for detection of maternal or fetal nutrient insufficiencies. When considering the potential consequences of growth restriction or poor nutrition for a fetus (including fetal morbidity and mortality), the need for more reliable assessments for growth restriction to supplement the archaic and highly unreliable assessment of fundal height is apparent. Application of the measurement of FGF21 in the blood of pregnant women could greatly improve clinical practice and the care obstetric patients receive.

Currently, there is no blood test for identification and/or diagnosis of fetal growth restriction during pregnancy. Without wishing to be bound by theory, measuring circulating FGF21 during pregnancy can improve the accuracy of detection of fetal growth restriction, as well as minimize the risk for growth restriction to be undetected. Furthermore this could be utilized as a screening test in order to refer an ‘at risk’ patient for further follow-up and clinical evaluation.

Data in human research subjects and in mouse models support this application of FGF21 as a biomarker for growth restriction and/or nutrient insufficiency in pregnancy. In both humans and mice, a low protein diet results in robustly increased serum FGF21 concentrations in pregnancy. Moreover, there are negative correlations between maternal, third trimester FGF21 and respective infant size, i.e. the higher the mother's FGF21 in late pregnancy, the smaller the infant in the first year of life. Finally, the growth restricted phenotype commonly observed in pups of dams fed low protein diets in pregnancy is absent in transgenic mice lacking FGF21 (FGF21 knock out mice). This finding suggests a direct role of FGF21 in fetal growth restriction. Considering together and without wishing to be bound by theory, FGF21 is a signal of nutrient insufficiency and the subsequent fetal growth restriction in both humans and mice.

### Example 2

Fibroblast Growth Factor 21 is a New Protein Sensor in Pregnancy

FGF21 in Pregnancy

Fibroblast growth factor 21 (FGF21) has not yet been studied for a potential role in developmental programming of future disease. Endocrine signals such as insulin, leptin, and adiponectin have been extensively investigated during pregnancy with aberrant effects on offspring growth and metabolic function. The aim of this study is to describe FGF21 in a healthy, pregnant population of women with overweight and obesity.

Between 35-36 weeks gestation, FGF21 was measured in fasting serum samples by ELISA, body composition by air displacement plethysmography, and food intake by self-reported Remote Food Photography Method in healthy women with overweight and obesity. Weight, length, and head circumference of infants of enrolled mothers were measured at birth, 1-2 months, 6 months and 12 months of age.

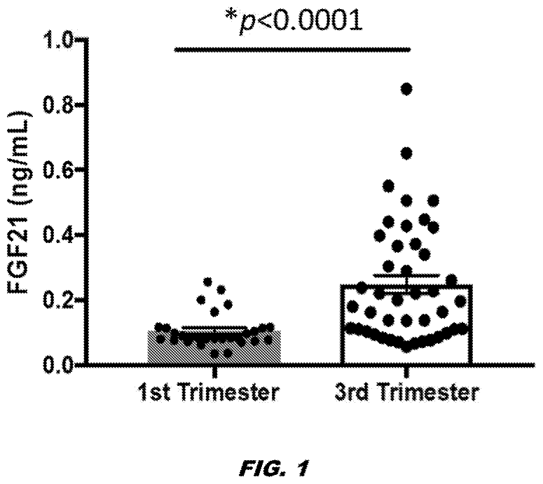

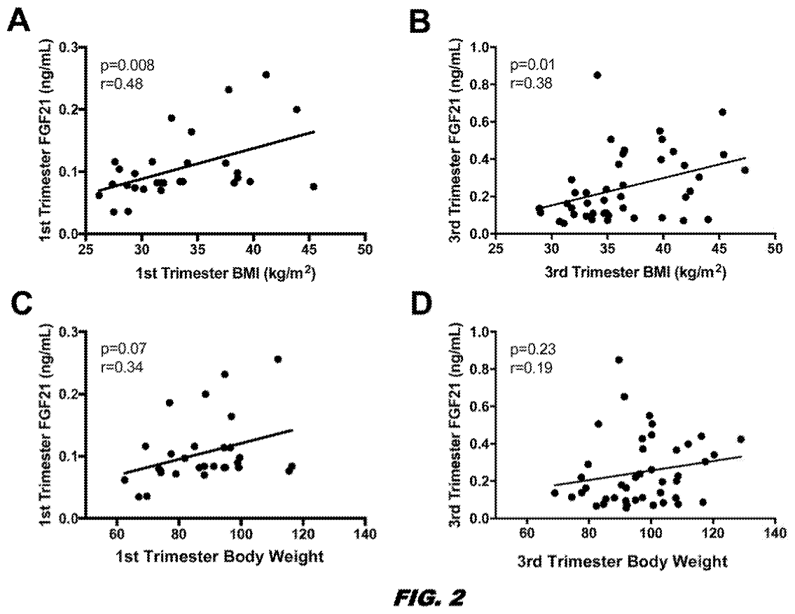

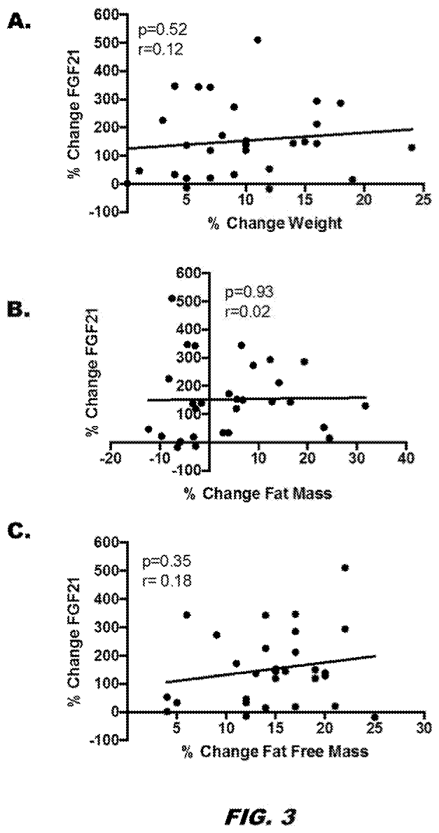

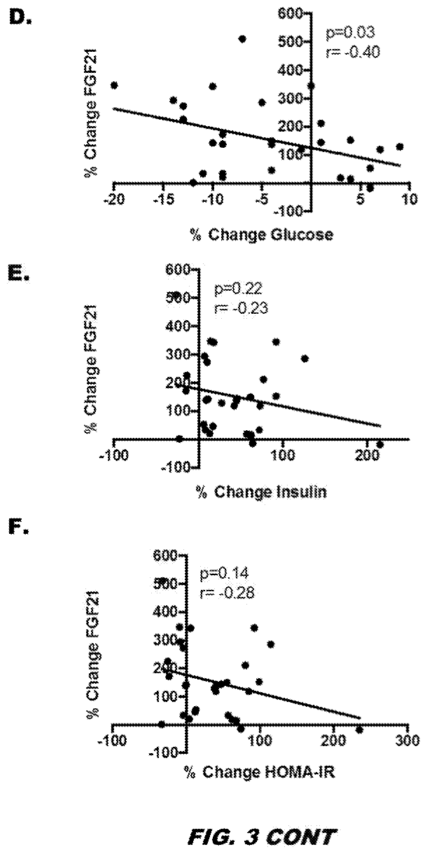

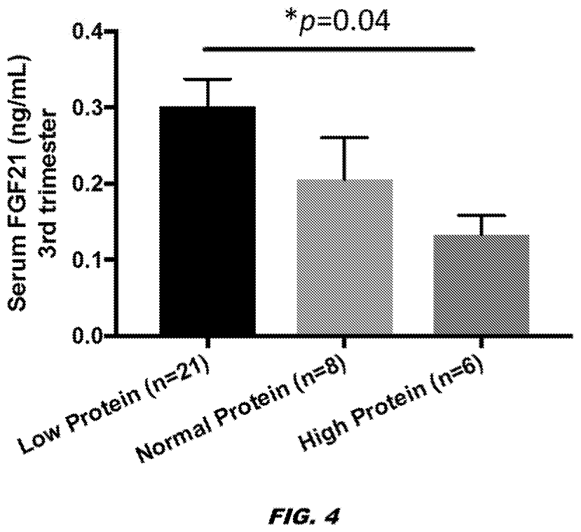

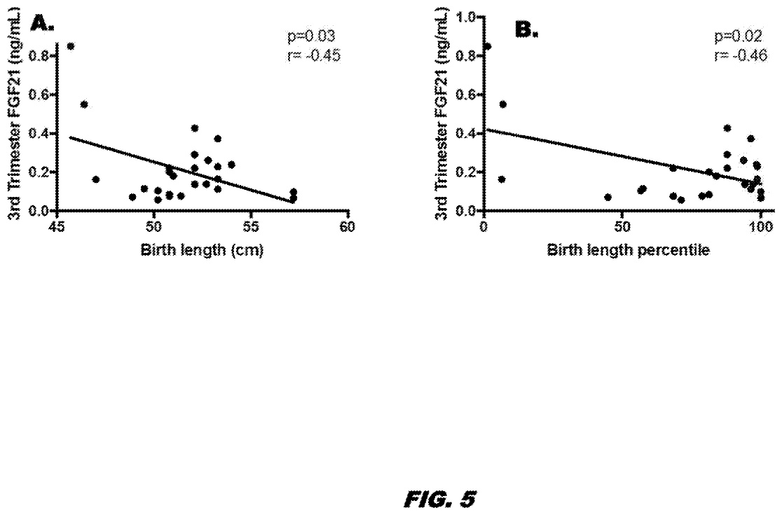

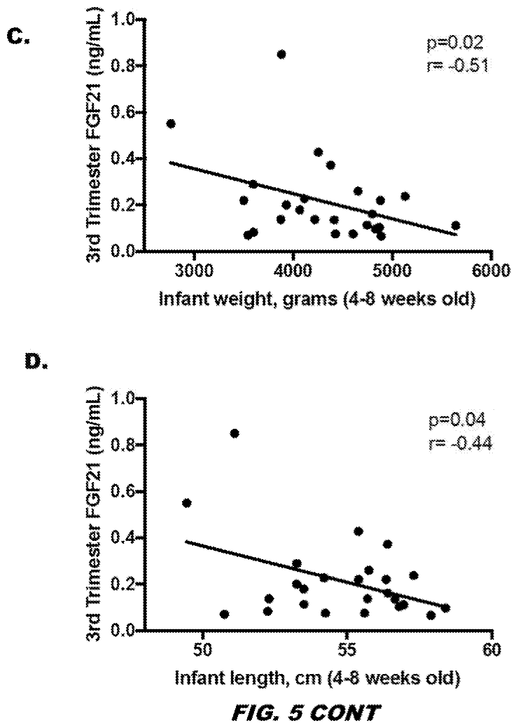

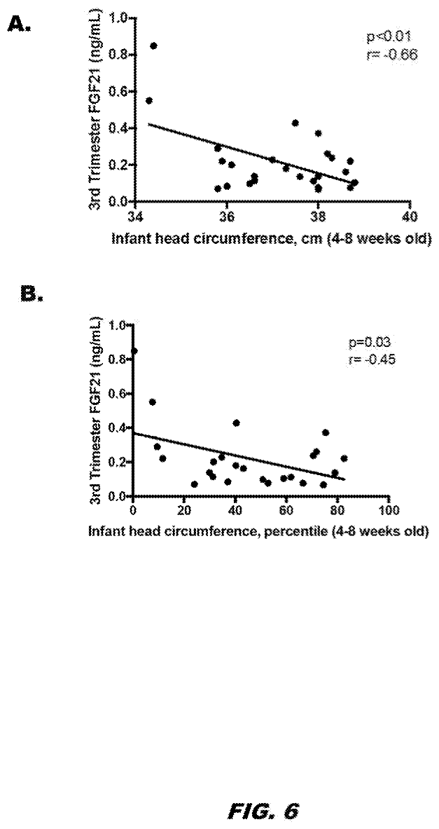

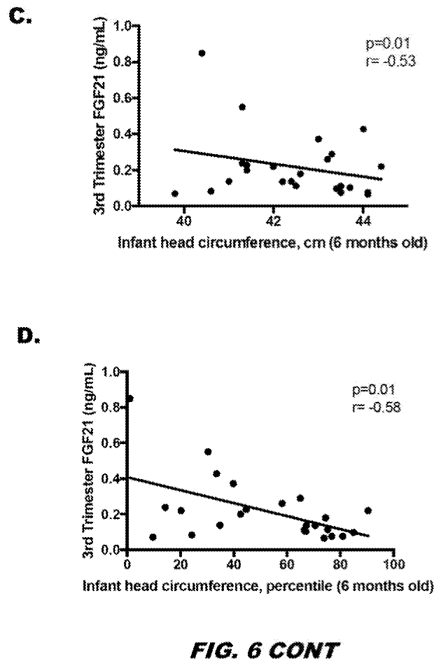

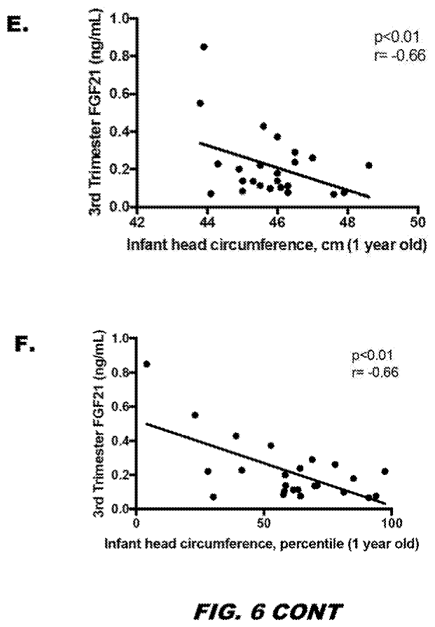

Results are displayed in FIG. 1-FIG. 6.

FGF21 is Required for Protein Sensing

FGF21 is thought to be a signal for protein restriction and has been shown to be robustly induced by low protein intake in mice, rats, and humans. Mice have been repeatedly shown to consume an average protein intake within a set point. However, the mechanisms responsible for this astute protein sensing are unknown. The purpose of this study is to test whether FGF21 is required for protein sensing in female mice.

Eight week old, female virgin C57BL/6 (n=20) and FGF21KO (n=19) mice were single housed and simultaneously provided a low (4% energy) and high (36 or 55% energy) protein diet and allowed to eat ad libitum. Food intake was measured daily for two weeks and protein intake calculated.

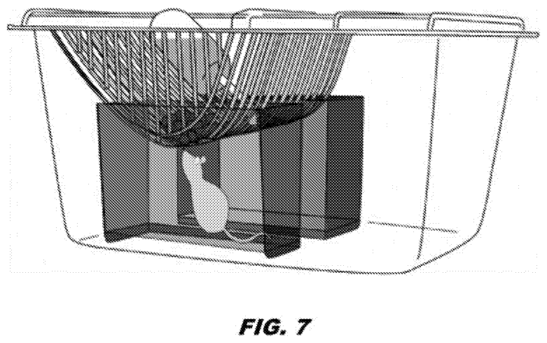

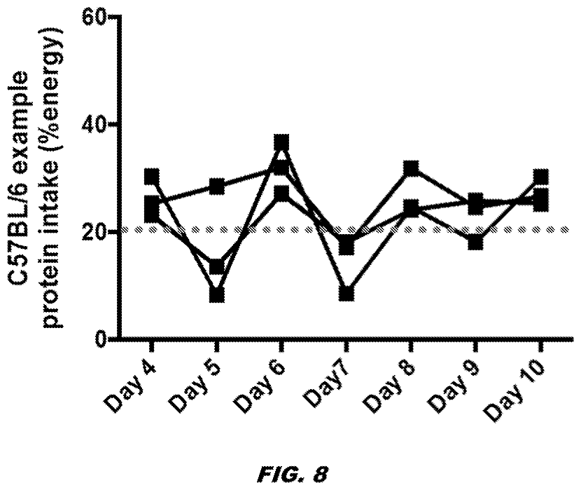

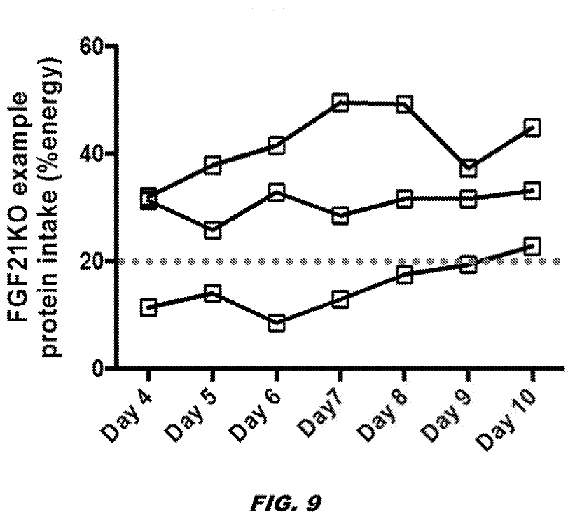

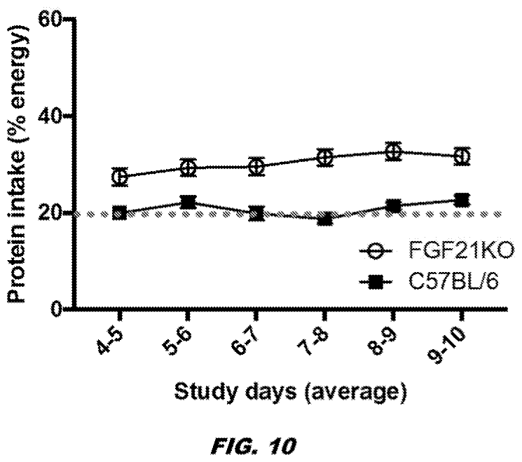

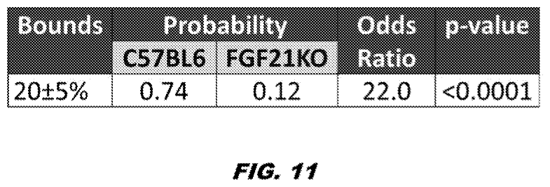

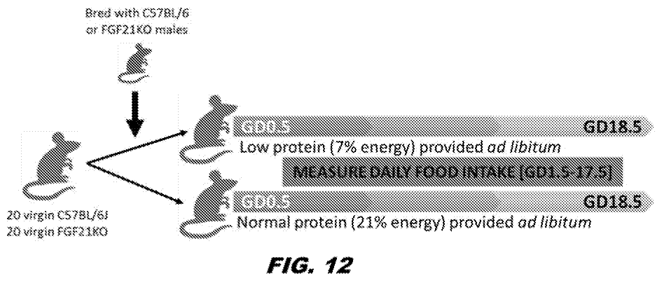

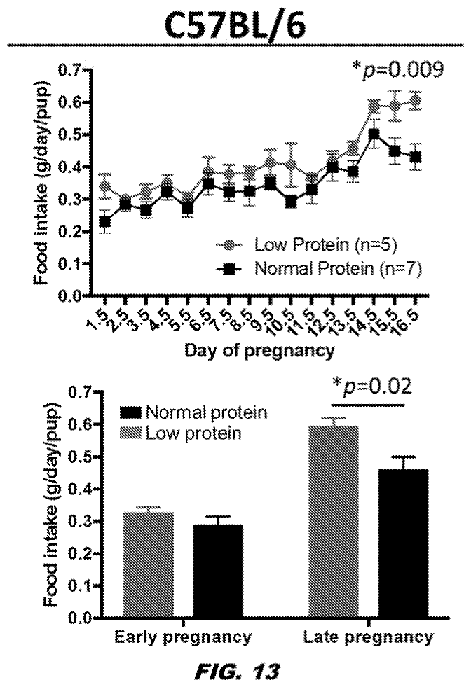

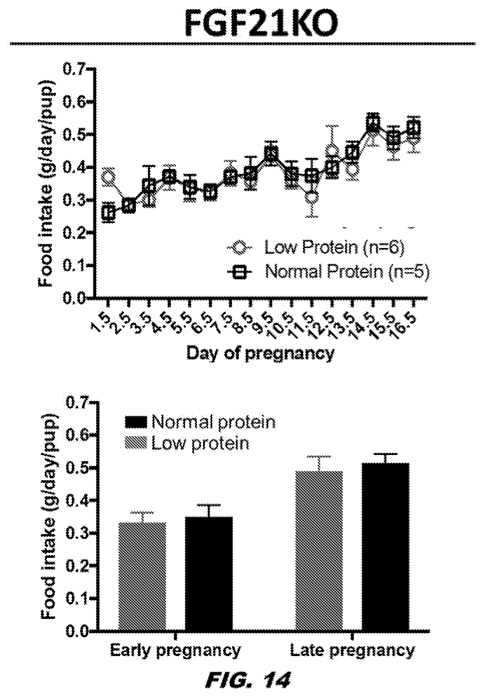

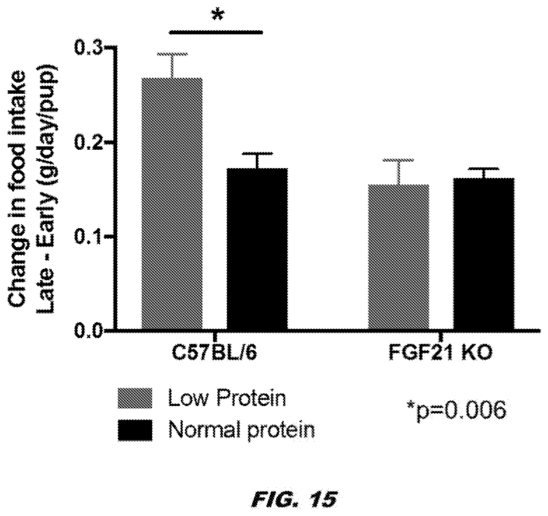

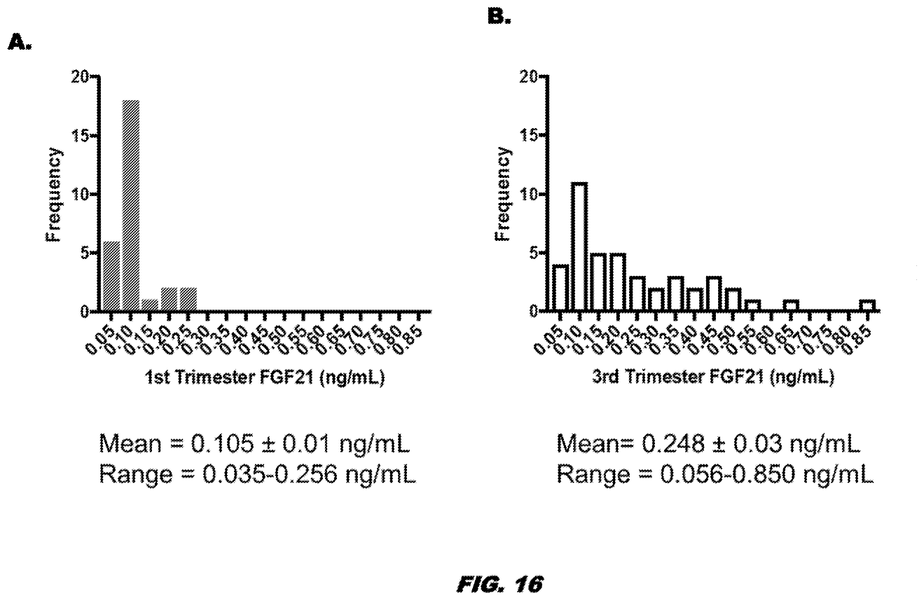

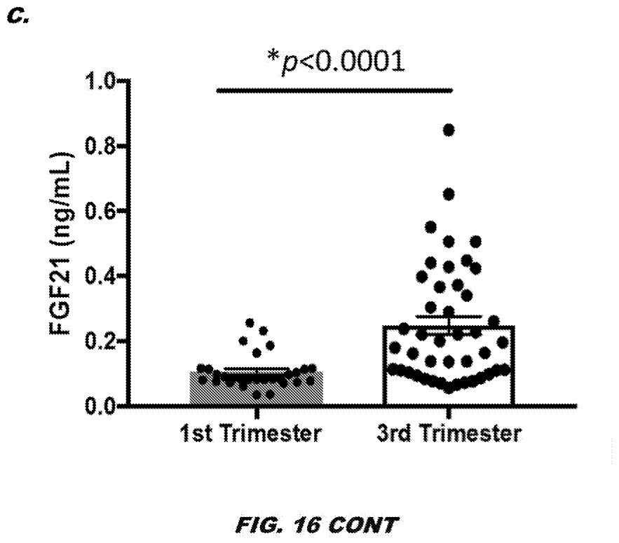

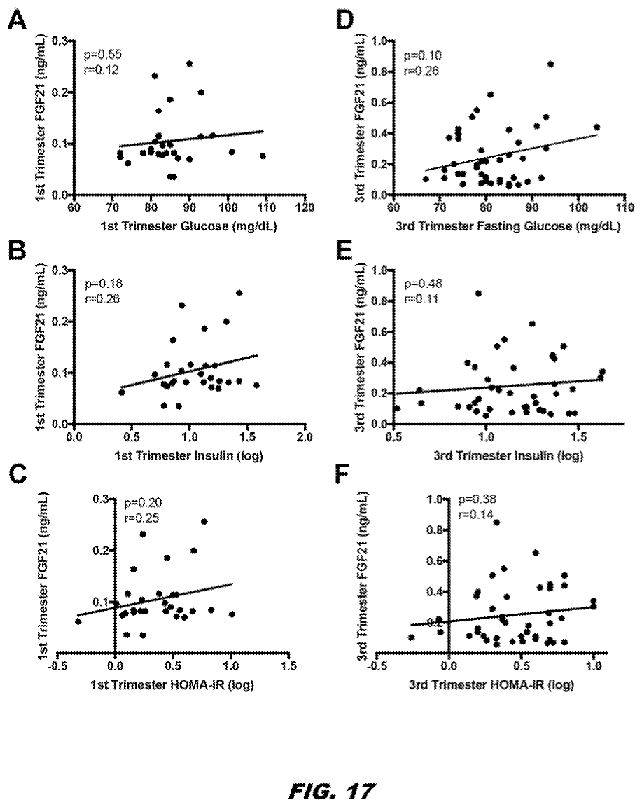

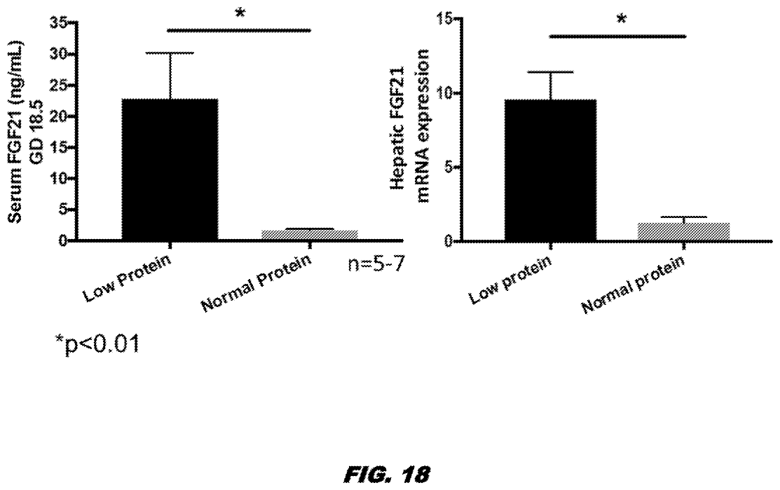

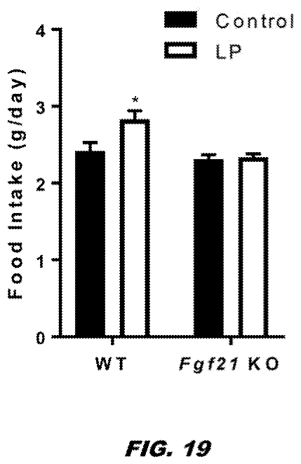

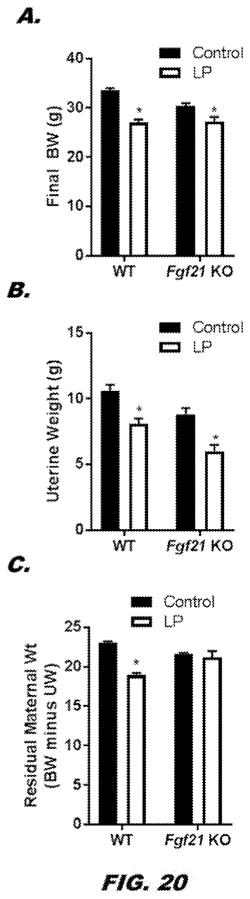

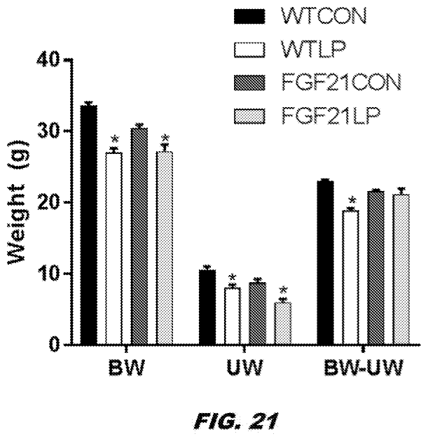

Results are displayed in FIG. 7-FIG. 11.

FGF21 and Protein Leverage

The Protein Leverage Hypothesis theorizes that total energy intake is driven by protein requirement, i.e. when consuming a low protein diet, an individual or animal will over-consume all macronutrients in order to satisfy daily protein balance and thereby increase total energy intake. The purpose of this study is to use the Protein Leverage Hypothesis as a model to test the role of FGF21 as a protein sensor in pregnancy.

Conclusions

In a healthy, pregnant population of women with overweight and obesity, serum FGF21 increases across pregnancy, positively correlates with adiposity, and is acutely regulated by macronutrient balance (i.e. glucose) in the third trimester.

Further, in a healthy, pregnant population of women with overweight and obesity, third trimester serum FGF21 negatively correlates with infant size at birth and growth during the first year of life.

Still further, FGF21 is required for protein sensing and subsequently regulation of protein intake in female, virgin mice.

Finally, FGF21 is required for the low-protein induced hyperphagia associated with protein leverage in pregnancy.

### Example 3

Fibroblast Growth Factor 21, Adiposity, and Macronutrient Balance in a Healthy, Pregnant Population with Overweight and Obesity

The regulation and actions of fibroblast growth factor 21 (FGF21) are responsive to energy status and macronutrient balance, and investigations of FGF21 in normal pregnancy, which could be informative for FGF21 biology, are seldom. The goal of our study was to examine FGF21 levels in a contemporary healthy, pregnant population. Without wishing to be bound by theory, FGF21 concentrations would be higher in women with increased adiposity, and FGF21 would increase commensurate with the increase in energy status (fat mass) and glucose intolerance (macronutrient balance) across pregnancy.

We phenotyped 43 women with overweight and obesity during pregnancy for weight, body composition, and fasting blood. Serum FGF21 was measured during the first and third trimesters. Placentas were collected at delivery.

Maternal FGF21 concentrations were positively correlated with body mass index and adiposity, but not lean mass or glucose homeostasis. FGF21 concentrations significantly increased from the first to third trimester of pregnancy (0.105 vs. 0.256 ng/mL, p<0.0001). Changes in FGF21 concentrations across pregnancy were not associated with changes in body weight or composition but inversely with the change in fasting glucose. FGF21 mRNA levels in placenta were very low and do not likely contribute to FGF21 in the maternal circulation.

FGF21 increases throughout pregnancy in our healthy cohort with overweight and obesity, independent of the placenta, and does not appear to be sensing the changes in energy balance (reflected in the change in maternal energy stores), but changes in macronutrient status. Thus, without wishing to be bound by theory, FGF21 may be a potential signal of maternal nutrient status in pregnancy.

Introduction

Fibroblast growth factor 21 (FGF21) is secreted in response to energy imbalance for the regulation of energy and nutrient metabolism. First discovered as a glucose sensitizer of adipose tissue, FGF21 has since been shown in both animal models and human studies to regulate glucose and lipid metabolism under various states of energy balancel. Metabolically challenged transgenic models overexpressing FGF21 and pharmacological administration of FGF21 to obese and diabetic animals and humans have revealed FGF21 to be capable of reducing body weight, protecting against diet induced obesity, improving glucose tolerance, and improving lipid profiles 1-7. However, cross sectional studies of the human population show that FGF21 is elevated in clinical conditions of energy or nutrient excess, such as obesity and type 2 diabetes 8-15. FGF21 is also positively correlated with metabolically unfavorable characteristics, such as hyperinsulinemia, insulin resistance, hypertriglyceridemia, and total cholesterol 11,14,16-22.

Pregnancy is a state of energy flux with energy and macronutrient demand increasing at variable rates and in response to various cues throughout gestation. Considering the impact of energy and macronutrient balance on FGF21 expression and action, understanding how FGF21 is regulated and/or acting in pregnancy, particularly in the presence of excess adiposity, could be highly informative to FGF21 biology. Conversely, given that adequate maternal nutrition (both over- and under-nutrition) is important for optimal pregnancy outcomes, FGF21 may serve as a new hormone to indicate patients at risk. Descriptions of FGF21 in the pregnant population are surprisingly limited to cross sectional studies in women with gestational diabetes or preeclampsia, with the majority of these studies showing FGF21 elevated in these conditions 22-26. These reports also confirm well established associations between FGF21 and an unfavorable metabolic milieu described in the non-pregnant state. For example, FGF21 in late pregnancy positively correlates with triglycerides and insulin resistance and inversely with adiponectin and HDL-cholesterol 22,23,25. The current knowledge of FGF21 biology in pregnancy is in need of more in depth studies throughout pregnancy. The aim of this study was to examine FGF21 levels in pregnant women in a contemporary cohort with overweight and obesity and to understand the role maternal energy stores and the placenta may play on maternal FGF21 secretion. Without wishing to be bound by theory, FGF21 concentrations may be higher in women with increased adiposity. Moreover, commensurate with the increase in energy status (fat mass) and glucose intolerance (macronutrient balance) across pregnancy, maternal FGF21 may increase throughout gestation.

Materials and Methods

Study Population

One hundred and fourteen pregnant women enrolled in the Expecting Success (n=54, NCT01610752) or the MomEE (n=60, NCT01954342) studies at Pennington Biomedical Research Center (PBRC) in Baton Rouge, Louisiana were potentially eligible for inclusion in this ancillary study. As detailed elsewhere, participants were recruited primarily from obstetrical offices, augmented by print and social media advertisements27. For entry into the parent studies, participants were required to be healthy, overweight or obese women (BMI>25kg/m2), aged 18-40 years with a single, viable, first trimester pregnancy (<14 weeks gestation). Patients were excluded from the parent studies for pregnancy-related conditions (known fetal anomaly, planned termination of pregnancy or adoption of infant, history of ≥3 consecutive miscarriages), pre-existing hypertension or diabetes (diagnosis prior to pregnancy, elevated HbA1c, or first trimester OGTT diagnosis of diabetes), psychological criteria (history or current psychotic disorder, current major depressive episode, bipolar disorder, history of anorexia or bulimia, current eating disorder, actively suicidal), medications (metformin, systemic steroids, antipsychotic agents, anti-seizure medications, or medications for ADHD), HIV, severe anemia, contraindications to exercise28, prior or planned (within one year of expected delivery) bariatric surgery, or recent history of or current smoking, alcohol, or drug use.

Of the 114 participants in the parent studies, 43 satisfied inclusion criteria for this ancillary study, provided consent for future use of biospecimens (i.e. blood and/or placenta), and had the required clinical data available at one or more time points for analysis. Briefly, the Expecting Success Study (NCT01610752) was an interventional study testing the efficacy of a lifestyle intervention designed to help overweight and obese pregnant women gain the recommended amount of weight in pregnancy. The MomEE Study (NCT01954342) was an observational study determining energy requirements of overweight and obese women across pregnancy. Both parent studies and this ancillary study were approved and monitored by the PBRC Institutional Review Board and all participants provided verbal and written consent prior to study initiation.

Clinic Assessments

For both the Expecting Success and MomEE Studies, study visits were performed in the first trimester (<16 weeks) and third trimester (35-36 weeks) in the morning following an overnight fast. Body weight, body composition, and blood measurements were collected in accordance with standard operating procedures of Pennington Biomedical Research Center to ensure scientific rigor and reproducibility.

Maternal Anthropometrics

Body weight was recorded twice with the participant fasting and wearing a hospital gown and undergarments only. The two recorded weights were averaged and the hospital gown weight subtracted. Body mass index was calculated as body weight (kg) divided by the square of study-measured height (m2). Body composition was assessed by air displacement plethysmography using a BOD POD® (COSMED, Chicago, Ill.) and fat mass and fat free mass were calculated from body volume as determined by the BOD POD® using equations developed by van Raaij et al. as previously validated 29,30.

Blood Chemistry

Serum glucose and insulin were assayed with the Beckman Coulter DXC 600 Pro (Beckman Coulter Inc., Brea, Calif.). FGF21 was measured in duplicate by sandwich enzyme-linked immunosorbent assay (ELISA) according to manufacturer instructions (RD191108200R, Fibroblast Growth Factor Human ELISA, Biovendor, Brno, Czech Republic). Serum was diluted 1:2 (125 μL of serum in 125 μL of dilution buffer) before analysis. The detectable concentration by the assay is 0.03 ng/mL. The intra-assay coefficient of variation was 3.9%.

Placenta Collection and Quantitative Real Time PCR

Placenta samples were collected, dissected and frozen within two hours of delivery. Samples were dissected at four separate sites of the placental disc and stored by section (basal plate, villous tissue, and chorionic plate) at −80° C. until being thawed for study. Experiments in this study used villous tissue snap frozen within two hours of delivery and pooled from all four collection sites. Placental RNA was isolated from flash-frozen tissue with the RNeasy Mini Kit (QIAGEN). Samples were quantified by Nanodrop and all samples had 260:280 and 260:230 ratios greater than 1.75. A 2000 ng sample of RNA was reverse transcribed with the High Capacity cDNA Reverse Transcription kit (Applied Biosystems) into cDNA. Quantitative real-time PCR was performed with 20 ng of cDNA, 300 nM of each forward and reverse primer, and iTaq universal SYBR green master mix. The PCR protocol was performed on a 7900HT PCR Machine (ThermoFisher Scientific, Waltham, Mass.) beginning with one cycle at 95° C. for 10 minutes, then 40 cycles of 95° C. for 15 seconds and 59° C. for 1 minute, and ended with a dissociation curve analysis. Primer sequences unique for each target gene were designed with primer BLAST to span exon-exon junctions (Table 1). The geometric mean of SDHA and TBP was used as an endogenous control and data was analyzed by AACt method.

TABLE 1
Prime sequences.

| Gene | Forward | Reverse |
|---|---|---|
| FGF21 | TGGATCGCTCCACTTTGACC | GGGCTTCGGACTGGTAAACA |
| FGF21 | GCTTCGGACTGGTAAACATTG | GGAGTCAAGACATCCAGGTT |
| SDHA | CGGTCCATGACTCTGGAGAT | AGCGAAGATCATGGCTGTCT |
| TBP | TGCACAGGAGCCAAGAGTGAA | CACATCACAGCTCCCCACCA |

Statistical Analysis

Data are presented as mean±standard error. Fasting insulin and HOMA-IR were logarithmically transformed. Statistical significance was determined by Spearman's correlation or paired student's t-tests when appropriate. Tests were performed with significance level a=0.05, and findings considered significant when P<α.

Results

Participants

The study cohort was 29±5 years old at the time of enrollment and was comprised of participants who self-identified as Caucasian (n=37), black (n=5) and other (n=1) (Table 2). Using first measured study height and weight (<14 weeks gestation), 14 participants were overweight (BMI 25-29.9kg/m2) and 29 participants were obese (BMI≥30 kg/m2) with 16 classified as obese class I, 7 obese class II, and 6 obese class III. All participants were in good health at enrollment. During study participation, four participants were diagnosed with gestational diabetes mellitus (GDM) (three treated with glyburide and one with glipizide), two with preeclampsia (one receiving magnesium sulfate during labor), and one participant with both GDM and PE (treated with diet only and magnesium sulfate during labor respectively) (described in Supplement Table 1).

TABLE 2
Participant characteristics.

| | | | First | | Third | | ρ |
|---|---|---|---|---|---|---|---|
| | Prepregnancy | | trimester | | trimester | | value* |
| Age at enrollment | | | 29.1 | (5.0) | | | |
| Parity (0/1/2/3/4) | 20/20/0/2/1 | | | | | | |
| Race (n) | | | | | | | |
| Black | 5 | (12%) | | | | | |
| White | 37 | (86%) | | | | | |
| Other | 1 | (2%) | | | | | |
| BMI Class (n) | | | | | | | |
| Overweight | 13 | | 14 | | 2 | | |
| Obese-Class I | 17 | | 16 | | 17 | | |
| Obese-Class II | 8 | | 7 | | 14 | | |
| Obese-Class III | 5 | | 6 | | 10 | | |
| BMI (kg/m²) | 32.8 | (5.4) | 33.5 | (5.3) | 36.6 | (4.7) | <0.0001 |
| Weight (kg) | 86.8 | (14.5) | 88.6 | (14.5) | 96.9 | (13.3) | <0.0001 |

| Fat mass (kg) | 40.4 | (10.5) | 41.8 | (9.7) | 0.04 |
|---|---|---|---|---|---|
| Lean mass (kg) | 48.3 | (6.1) | 55.3 | (6.1) | <0.0001 |
| Fasting glucose | 84.6 | (8.0) | 81.5 | (7.6) | 0.002 |
| (mg/dL) | | | | | |
| Fasting insulin | 12.4 | (7.9) | 16.8 | (9.3) | <0.0001 |
| (mU/L) | | | | | |
| HOMA-IR | 2.7 | (2.0) | 3.4 | (2.1) | 0.002 |

Values reported as mean ± standard deviation.
*represents significant differences between first and third trimesters.

Pattern of FGF21 in Human Pregnancy

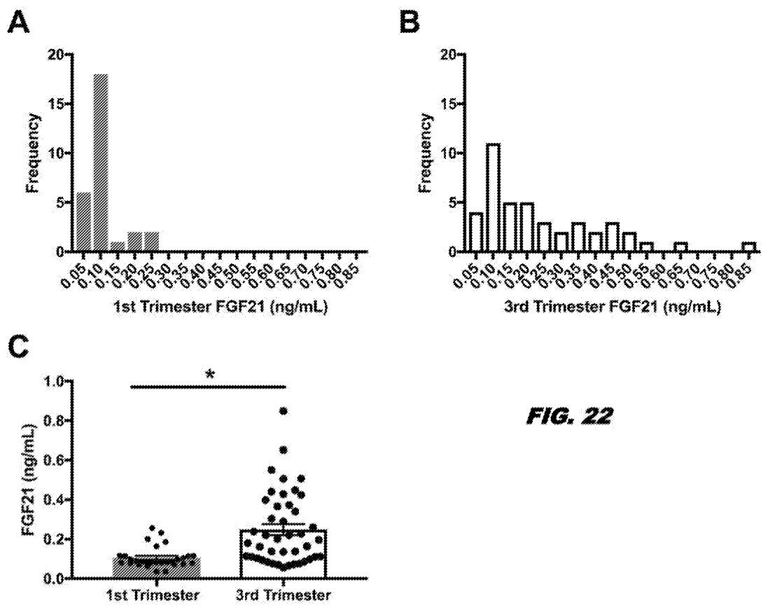

In fasting serum samples collected from healthy women in the first and third trimester of pregnancy, FGF21 concentrations were found to be highly variable. In the first trimester (n=29), FGF21 concentrations ranged from 0.035 to 0.256 ng/mL (FIG. 22, panel A) and an even larger degree of variability was observed in the third trimester (n=43, range 0.056-0.850 ng/mL, FIG. 22, panel B). First trimester FGF21 concentrations showed ten-fold less variance compared to third trimester concentrations, 0.003 versus 0.033 respectively (p<0.0001). For participants with FGF21 concentrations measured at both the first and third trimesters (n=29), FGF21 concentrations increased across pregnancy and were more than 2-fold higher in the third trimester of pregnancy (35-36 weeks) compared to concentrations measured in the first trimester (<16 weeks), 0.256 vs. 0.105 ng/mL respectively (FIGS. 22, panel C and D).

FGF21 is Correlated with Maternal Body Size and Adiposity Throughout Pregnancy

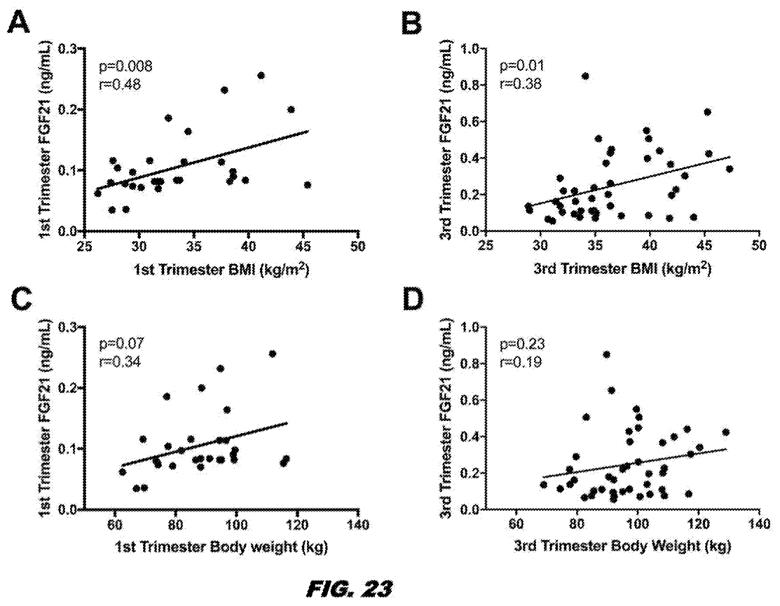

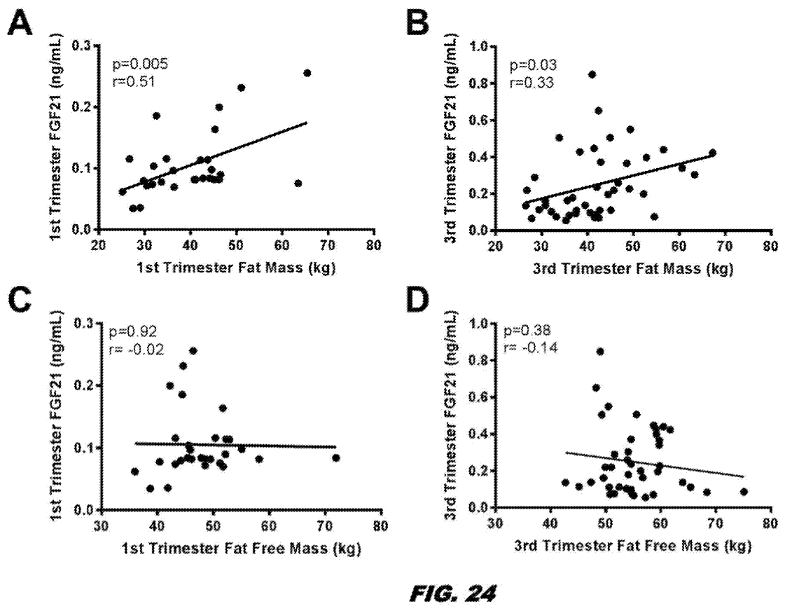

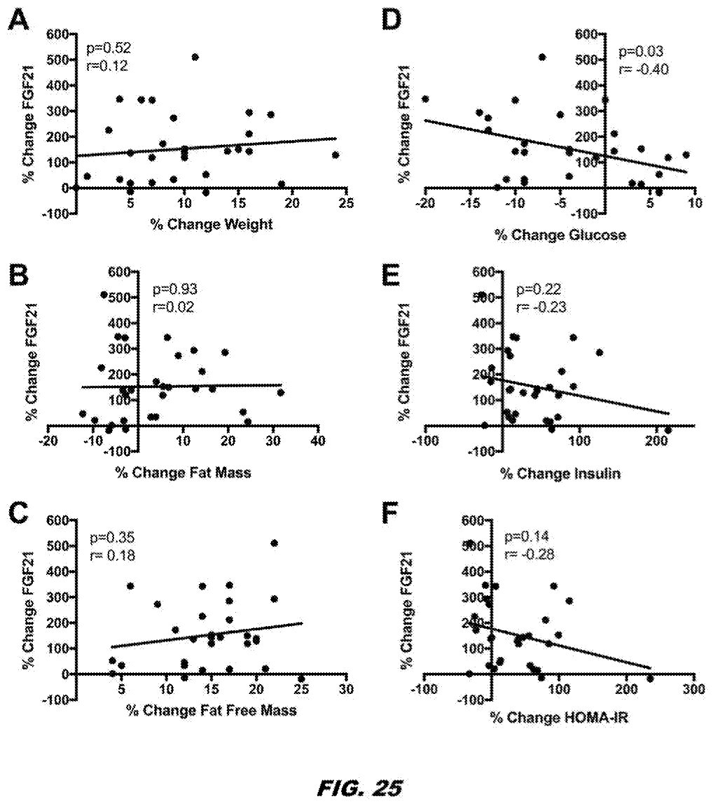

In non-pregnant individuals, FGF21 is elevated with higher BMI and increased adiposity 14. In the pregnant state, we found FGF21 concentrations are significantly and positively correlated with maternal BMI in both the first (ρ=0.48, p=0.008) and third trimesters (ρ=0.40, p=0.008) (FIGS. 23, panels A and B). However, FGF21 correlated to body weight in the first trimester only (ρ=0.39, p=0.04). FGF21 concentrations are also strongly and significantly correlated with maternal adiposity reported as total fat mass (kg) in both the first and third trimesters (1st trimester: ρ=0.47, p=0.01; 3rd trimester: ρ=0.39, p=0.01) (FIGS. 23, panels C and 2D). FGF21 concentration was not significantly correlated with maternal fat free mass in either trimester (1st trimester: ρ=0.21, p=0.26, 3rd trimester: ρ=−0.06, p=0.68).

FGF21 is not Correlated with Glucose Homeostasis in Normoglycemic Pregnancy

FGF21 has been shown to positively correlate with fasting glucose, fasting insulin, and insulin resistance in animal models and cross sectional, non-pregnant human studies 8,11,16-18. In our population of pregnant women with majority normal glucose tolerance and maternal overweight or obesity, no significant relationships between FGF21 and fasting glucose (p=0.61 and 0.63 respectively), fasting insulin (p=0.23 and 0.54 respectively), or HOMA-IR (p=0.24 and 0.52 respectively) were observed in either the first or third trimester.

Change in Glucose, not AdiposityC correlates with Change in FGF21 Across Pregnancy

In an effort to understand if alterations in maternal characteristics throughout gestation may explain the increase in FGF21, we tested for relationships between the change in FGF21 (absolute and percent) and change in known contributors of FGF21 in the non-pregnant state, namely adiposity and metabolic status. Body weight, fat mass, fat free mass, and fasting insulin each increased from the first to the third trimester, although fasting glucose marginally decreased (Table 2). Although we observed significant relationships with body composition, namely adiposity, at single time points, the changes in these parameters across pregnancy were not associated with the changes in FGF21 concentrations measured across the same time interval (Table 3). However, the change in FGF21 concentrations was significantly and inversely correlated with the change in fasting glucose (ρ=−0.44, p=0.02). No relationship was observed between changes in FGF21 and alterations in insulin or HOMA-IR (ρ=−0.16, p=0.40 and ρ=−0.26, p=0.18 respectively).

TABLE 3
Spearman correlations between percent change in
FGF21 and percent change in body size, composition and
macronutrient balance from the first to third trimesters.

| | ρ | ρ value |
|---|---|---|
| Change in body weight | 0.17 | 0.38 |
| Change in BMI | 0.17 | 0.38 |
| Change in fat mass | 0.16 | 0.41 |
| Change in fat free mass | 0.14 | 0.48 |
| Change in glucose | −0.44 | 0.02 |
| Change in insulin | −0.16 | 0.40 |
| Change in HOMA-IR | −0.26 | 0.18 |

The Placenta is Not a Primary Source of FGF21 Production in Pregnancy

Considering the increase in FGF21 is not correlated with changes in maternal energy stores and considering previous studies 24,31,32 that suggest FGF21 may be secreted by the placenta, the increase in FGF21 concentrations across pregnancy can originate from products of conception (i.e. placenta and fetus). Utilizing two different sets of primers including the primers reported in past studies31, we were unable to detect meaningful and replicable quantities of FGF21 transcript in human placenta by qPCR. These observations are not due to poor sample quality or error in qPCR protocol. The RNA extraction protocol used results in a RIN of 7 in human placenta; moreover, we were able to measure over 20 additional genes (including housekeeping genes SDHA and TBP) with resulting CTs based on published findings in human placenta.

Discussion

In this study, we report FGF21 levels in a pregnant population with pregravid overweight or obesity. The three observations from this work are that circulating FGF21 measured in fasting conditions is correlated with maternal body mass index and adiposity at cross sectional time points throughout pregnancy, FGF21 concentrations increase across pregnancy, and this increase in FGF21 does not appear to be coming from the placenta or a change in maternal energy stores but with alterations in maternal glucose concentrations.

The relationship of FGF21, BMI, and adiposity has been well reported in non-pregnant animal models and human populations 10,14,15,18. In this study, we demonstrate FGF21 concentrations positively correlate with maternal body mass index and adiposity in pregnant women in both the first and third trimesters. Considering pregnancy is a time for increased energy deposition in preparation for postpartum energy demands, e.g. lactation, these findings show the relationship between FGF21 and adiposity observed in non-pregnant states holds in cross sectional analysis performed at different time points across pregnancy. However, it is important to note the individual changes in adiposity across pregnancy did not predict the variations in FGF21 concentrations. These data suggest that the physiological drivers for deposition of maternal energy in fat mass are independent of changes in FGF21.

Notably, we were unable to confirm that high concentrations of FGF21 are present in pregnant individuals with increasing levels of glucose intolerance. Lack of support for this relationship in our population could be due to insufficient statistical power because we excluded participants with pregravid diabetes and impaired glucose tolerance in their first trimester (HbA1c>6.5% and/or failed 75 g oral glucose tolerance test). Although five participants were diagnosed with GDM during study participation, this initial exclusion likely limited the variability in glucose dysfunction within our cohort (fasting glucose ranged 72-109 mg/dL) despite the large range of maternal BMI (25.6 to 45.4 kg/m2). While fasting insulin and HOMA-IR showed moderate distribution among participants across pregnancy (see Table 2), glucose regulation appeared well controlled. Indeed, the percent change in fasting glucose (from the first to third trimester) ranged from −20% to +9%, with fasting glucose decreasing in two-thirds of participants. Therefore, there was limited variability in measures of glucose homeostasis for comparison to FGF21. Future studies need to be conducted in pregnant women with varying degrees of glucose tolerance to further elucidate the role of FGF21 in response to the worsened maternal glucose homeostasis throughout pregnancy.

We observed FGF21 concentrations were less variable and significantly lower in the first trimester compared to the third trimester of pregnancy. Considering FGF21 has been reported to be produced by the human placenta by two separate laboratories (in one study by ELISA of placenta explant media 24 and two other studies by qPCR of flash frozen human placenta samples 31,32), the increase in circulating FGF21 can be attributed to placenta tissue. Although, after extensive qPCR experiments in term human placenta from a subset of our cohort (n=19), we were unable to quantify expression of FGF21 in the samples studied despite detection of other genes such as SDHA and TBP. We are therefore confident the human placenta is not contributing meaningful amounts of FGF21 into the maternal circulation. Instead, considering evidence of robust elevation in hepatic FGF21 mRNA throughout pregnancy recently reported in wild-type mice 33, we posit the elevation in circulating FGF21 observed in our human cohort is likely resultant from the liver. Moreover, there is extensive evidence showing FGF21 synthesis and secretion by adipocytes in rodents and humans, and it may also be likely that maternal adipocytes are contributing in part to this FGF21 elevation considering the increase in fat mass across pregnancy 14,34.

FGF21 has been regularly shown to correlate with glucose homeostasis in non-pregnant and pregnant populations with glucose dysfunction which could be another possible explanation for the change in FGF21 across pregnancy. Despite the limited variability of glucose homeostasis within our cohort, we observed a significant negative correlation between alterations in FGF21 and changes in fasting glucose. Of note, there was not a significant correlation between change in insulin or HOMA-IR and change in FGF21. The original role elucidated for FGF21 was as a glucose regulator, however the relationship between FGF21 and glucose regulation in pregnancy merits further investigation as we made these observations within a healthy population with little variability.

Longitudinal measurements of FGF21, body composition assessments in pregnancy by BOD POD®, as well as the availability of archived placenta tissue provide strength to our study. However, our study does have limitations. First is our small sample size. In an effort to maximize resources, we leveraged existing data and archived biospecimens from two parent studies (totaling n=114 at the time of analysis) for the study. Considering the necessary data and biospecimens to answer our question (i.e. archived third trimester serum, data collected in the first and third trimesters, and consent for future use of biospecimens and data), only 43 of the 114 enrolled participants were eligible for this ancillary. Furthermore, our sample is primarily made up of Caucasian women and exclusively of women with overweight and obesity. Indeed, replication of these results in a larger and more diverse cohort as well as a lean cohort would be advantageous next steps. Second, 21 of 43 subjects participated in a lifestyle intervention aimed to attenuate gestational weight gain during pregnancy. We conducted additional analyses to investigate the potential influence of the intervention and found no differences across pregnancy for individuals receiving or not receiving the intervention, including change in FGF21, weight, BMI, fat mass, fat free mass, glucose, insulin, or HOMA-IR (Supplement Table 3). There were significant differences in BMI, weight, and fat mass at enrollment between individuals receiving the intervention compared to no intervention (Supplement Table 2). However, our “no intervention” group is a compilation of a subset of the control group from the Expecting Success Study (n=6) whose inclusion criteria was BMI≥25kg/m² and participants from the MomEE Study (n=16) which did not have an intervention and whose inclusion criteria BMI≥30 kg/m².

Since the changes in FGF21 were associated with changes in fasting glucose and not with the changes in body energy stores (i.e. weight or fat mass), variations in acute energy status, such as with the maternal diet that influence both tissue deposition and glucose regulation, might be involved. In summary, we observed maternal FGF21 is positively correlated with maternal body mass index and adiposity before and throughout pregnancy, and FGF21 is likely responsive to short-term changes in macronutrient balance induced by maternal diet rather than long-term changes in energy balance reflected in the maternal energy stores.

Supplement Table 1: Participant Characteristics by Pregnancy History

| | 1^{st} Trimester | | | | 3^{rd} Trimester | | | |
|---|---|---|---|---|---|---|---|---|
| | No complications | GDM | PE | | No complications | GDM | PE | |
| | (n = 36) | (n = 5) | (n = 3) | p value | (n = 36) | (n = 5) | (n = 3) | p value |
| BMI (kg/m²) | 33.3 (4.9) | 34.6 (7.7) | 30.6 (7.0) | 0.59 | 36.4 (4.4) | 37.4 (6.8) | 35.8 (5.4) | 0.87 |
| Weight (kg) | 88.3 (13.2) | 92.6 (21.5) | 77.1 (19.5) | 0.34 | 96.5 (12.7) | 100.0 (19.4) | 90.2 (16.0) | 0.62 |
| Fasting glucose (mg/dL) | 84 (7) | 93 (13) | 82 (6) | 0.05 | 80 (6) | 79 (7) | 78 (5) | 0.26 |
| Fasting insulin (mU/L) | 11.7 (6.8) | 19.3 (13.1) | 8.8 (3.6) | 0.09 | 16.0 (8.8) | 23.2 (12.5) | 12.1 (3.0) | 0.19 |
| HOMA-IR | 2.5 (1.6) | 4.7 (3.8) | 1.8 (0.8) | 0.04 | 3.3 (2.0) | 5.0 (2.9) | 2.3 (0.6) | 0.14 |

Values reported as mean ± standard deviation,
PE = Preeclampsia,
GDM = gestational diabetes mellitus
Note: one participant was diagnosed with both GDM and PE

SUPPLEMENT TABLE 2
Enrollment characteristics by intervention group

| | | | Lifestyle GWG | | |
|---|---|---|---|---|---|
| | Control | | Intervention | | |
| | (n = 22) | | (n = 21) | | p value* |
| Body mass index (kg/m²) | 35.1 | (5.4) | 31.7 | (4.6) | 0.03 |
| Fat mass (kg) | 43.2 | (11.0) | 37.4 | (9.2) | 0.07 |
| Lean mass (kg) | 50.6 | (6.6) | 45.9 | (4.5) | 0.01 |
| Fasting glucose (mg/dL) | 88.1 | (8.9) | 81.0 | (5.0) | 0.002 |
| Fasting insulin (mU/L) | 16.1 | (8.9) | 8.6 | (4.3) | 0.001 |
| HOMA-IR | 3.64 | (2.36) | 1.73 | (0.87) | 0.001 |
| FGF21 (ng/mL) | 0.113 | (0.053) | 0.092 | (0.053) | 0.295 |

Values reported as mean ± standard deviation;
For BMI, body weight, fat mass and lean mass: n = 22 for control and 21 for intervention; for glucose, insulin and HOMA-IR: n = 21 for control and 21 for intervention; for FGF21: n = 18 for control and 11 for intervention

SUPPLEMENT TABLE 3
Change in characteristics across pregnancy
(from first to third trimester) by intervention group

| | | | Lifestyle GWG | | |
|---|---|---|---|---|---|
| | Control | | Intervention | | |
| | (n = 22) | | (n = 21) | | p value* |
| Weight (kg) | 8.4 | (5.0) | 8.3 | (4.2) | 0.906 |
| Body mass index (kg/m²) | 3.1 | (1.8) | 3.1 | (1.6) | 0.955 |
| Fat mass (kg) | 1.9 | (4.6) | 0.9 | (4.2) | 0.444 |
| Lean mass (kg) | 6.6 | (3.5) | 7.5 | (2.1) | 0.324 |
| Fasting glucose (mg/dL) | −4.4 | (7.3) | −2.1 | (5.7) | 0.262 |
| Fasting insulin (mU/L) | 3.5 | (6.1) | 5.0 | (5.5) | 0.406 |
| HOMA-IR | 0.50 | (1.6) | 0.93 | (1.15) | 0.319 |
| FGF21 (ng/mL) | 0.162 | (0.147) | 0.131 | (0.105) | 0.551 |

Values reported as mean ± standard deviation;
For BMI, body weight, fat mass and lean mass: n = 22 for control and 21 for intervention; for glucose, insulin and HOMA-IR: n = 21 for control and 21 for intervention; for FGF21: n = 18 for control and 11 for intervention

### References Cited in this Example

1. Kharitonenkov A, Shiyanova T L, Koester A, et al. FGF-21 as a novel metabolic regulator. J Clin Invest. 2005; 115(6):1627-1635.

2. Coskun T, Bina H A, Schneider M A, et al. Fibroblast growth factor 21 corrects obesity in mice. Endocrinology. 2008; 149(12):6018-6027.

3. Xu J, Lloyd D J, Hale C, et al. Fibroblast growth factor 21 reverses hepatic steatosis, increases energy expenditure, and improves insulin sensitivity in diet-induced obese mice. Diabetes. 2009; 58(1):250-259.

4. Camporez J P, Jornayvaz F R, Petersen M C, et al. Cellular mechanisms by which FGF21 improves insulin sensitivity in male mice. Endocrinology. 2013; 154(9):3099-3109.

5. Kharitonenkov A, Wroblewski V J, Koester A, et al. The metabolic state of diabetic monkeys is regulated by fibroblast growth factor-21. Endocrinology. 2007; 148(2):774-781.

6. Gaich G, Chien J Y, Fu H, et al. The effects of LY2405319, an FGF21 analog, in obese human subjects with type 2 diabetes. Cell Metab. 2013; 18(3):333-340.

7. Wente W, Efanov A M, Brenner M, et al. Fibroblast growth factor-21 improves pancreatic beta-cell function and survival by activation of extracellular signal-regulated kinase ½ and Akt signaling pathways. Diabetes. 2006; 55(9):2470-2478.

8. Chavez A O, Molina-Carrion M, Abdul-Ghani M A, Folli F, Defronzo R A, Tripathy D. Circulating fibroblast growth factor-21 is elevated in impaired glucose tolerance and type 2 diabetes and correlates with muscle and hepatic insulin resistance. Diabetes Care. 2009; 32(8):1542-1546.

9. Chen W W, Li L, Yang G Y, et al. Circulating FGF-21 levels in normal subjects and in newly diagnose patients with Type 2 diabetes mellitus. Exp Clin Endocrinol Diabetes. 2008; 116(1):65-68.

10. Mraz M, Bartlova M, Lacinova Z, et al. Serum concentrations and tissue expression of a novel endocrine regulator fibroblast growth factor-21 in patients with type 2 diabetes and obesity. Clin Endocrinol (Oxf). 2009; 71(3):369-375.

11. Lin Z, Wu Z, Yin X, et al. Serum levels of FGF-21 are increased in coronary heart disease patients and are independently associated with adverse lipid profile. PLoS One. 2010; 5(12):e15534.

12. Chen C, Cheung B M, Tso A W, et al. High plasma level of fibroblast growth factor 21 is an Independent predictor of type 2 diabetes: a 5.4-year population-based prospective study in Chinese subjects. Diabetes Care. 2011; 34(9):2113-2115.

13. Zhang Y, Xie Y, Berglund E D, et al. The starvation hormone, fibroblast growth factor-21, extends lifespan in mice. Elife. 2012; 1:e00065.

14. Zhang X, Yeung D C, Karpisek M, et al. Serum FGF21 levels are increased in obesity and are independently associated with the metabolic syndrome in humans. Diabetes. 2008; 57(5):1246-1253.

15. Dushay J, Chui P C, Gopalakrishnan G S, et al. Increased fibroblast growth factor 21 in obesity and nonalcoholic fatty liver disease. Gastroenterology. 2010; 139(2):456-463.

16. Li L, Yang G, Ning H, Yang M, Liu H, Chen W. Plasma FGF-21 levels in type 2 diabetic patients with ketosis. Diabetes Res Clin Pract. 2008; 82(2):209-213.

17. Li K, Li L, Yang M, Zong H, Liu H, Yang G. Effects of rosiglitazone on fasting plasma fibroblast growth factor-21 levels in patients with type 2 diabetes mellitus. Eur J Endocrinol. 2009; 161(3):391-395.

18. Tan BK, Hallschmid M, Adya R, Kern W, Lehnert H, Randeva H S. Fibroblast growth factor 21 (FGF21) in human cerebrospinal fluid: relationship with plasma FGF21 and body adiposity. Diabetes. 2011; 60(11):2758-2762.

19. Stein S, Stepan H, Kratzsch J, et al. Serum fibroblast growth factor 21 levels in gestational diabetes mellitus in relation to insulin resistance and dyslipidemia. Metabolism. 2010; 59(1):33-37.

20. Gallego-Escuredo J M, Gomez-Ambrosi J, Catalan V, et al. Opposite alterations in FGF21 and FGF19 levels and disturbed expression of the receptor machinery for endocrine FGFs in obese patients. Int J Obes (Lond). 2015; 39(1):121-129.

21. Li H, Bao Y, Xu A, et al. Serum fibroblast growth factor 21 is associated with adverse lipid profiles and gamma-glutamyltransferase but not insulin sensitivity in Chinese subjects. J Clin Endocrinol Metab. 2009; 94(6):2151-2156.

22. Wang D, Zhu W, Li J, An C, Wang Z. Serum concentrations of fibroblast growth factors 19 and 21 in women with gestational diabetes mellitus: association with insulin resistance, adiponectin, and polycystic ovary syndrome history. PLoS One. 2013; 8(11):e81190.

23. Stepan H, Kley K, Hindricks J, et al. Serum levels of the adipokine fibroblast growth factor-21 are increased in preeclampsia. Cytokine. 2013; 62(2):322-326.

24. Tan B K, Sivakumar K, Bari M F, Vatish M, Randeva H S. Lower cerebrospinal fluid/plasma fibroblast growth factor 21 (FGF21) ratios and placental FGF21 production in gestational diabetes. PLoS One. 2013; 8(6):e65254.

25. Li S M, Wang W F, Zhou L H, et al. Fibroblast growth factor 21 expressions in white blood cells and sera of patients with gestational diabetes mellitus during gestation and postpartum. Endocrine. 2015; 48(2):519-527.

26. Megia A, Gil-Lluis P, Naf S, et al. Cord blood FGF21 in gestational diabetes and its relationship with postnatal growth. Acta Diabetol. 2015; 52(4):693-700.

27. Sutton E F, Cain L E, Vallo P M, Redman L M. Strategies for Successful Recruitment of Pregnant Patients Into Clinical Trials. Obstet Gynecol. 2017; 129(3):554-559.

28. ACOG Committee opinion. Number 267, January 2002: exercise during pregnancy and the postpartum period. Obstet Gynecol. 2002; 99(1):171-173.

29. Marshall N E, Murphy E J, King J C, et al. Comparison of multiple methods to measure maternal fat mass in late gestation. Am J Clin Nutr. 2016; 103(4):1055-1063.

30. van Raaij J M, Peek M E, Vermaat-Miedema S H, Schonk C M, Hautvast J G. New equations for estimating body fat mass in pregnancy from body density or total body water. Am J Clin Nutr. 1988; 48(1):24-29.

31. Dekker Nitert M, Barrett H L, Kubala M H, et al. Increased placental expression of fibroblast growth factor 21 in gestational diabetes mellitus. J Clin Endocrinol Metab. 2014; 99(4):E591-598.

32. Dekker Nitert M, Scholz-Romero K, Kubala M H, McIntyre H D, Callaway L K, Barrett H L. Placental fibroblast growth factor 21 is not altered in late-onset preeclampsia. Reprod Biol Endocrinol. 2015; 13:14.

33. Cui Y, Giesy S L, Hassan M, Davis K, Zhao S, Boisclair Y R. Hepatic FGF21 production is increased in late pregnancy in the mouse. Am J Physiol Regul Integr Comp Physiol. 2014; 307(3):R290-298.

34. Dutchak P A, Katafuchi T, Bookout A L, et al. Fibroblast growth factor-21 regulates PPARgamma activity and the antidiabetic actions of thiazolidinediones. Cell. 2012; 148(3):556-567.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A non-invasive method of identifying a fetus at risk for fetal growth restriction, the method comprising:
incubating a biological sample from a subject pregnant with a fetus with an agent that binds to FGF21 protein or a fragment thereof, wherein a FGF21-binding agent complex is formed;
measuring the amount of FGF21 bound agent in the biological sample obtained from the subject; and
identifying the fetus as at risk for fetal growth restriction when FGF21 protein levels in the sample are elevated above control levels.

2. A non-invasive method to identify a fetus with fetal growth restriction, the method comprising:
incubating a biological sample from a subject pregnant with a fetus with an agent that binds to FGF21 protein or a fragment thereof, wherein a FGF21-binding agent complex is formed;
measuring the amount of FGF21 bound agent in the biological sample obtained from the subject; and
identifying the fetus as having fetal growth restriction when FGF21 protein levels in the sample are elevated above control levels.

3. A non-invasive method of identifying a fetus exposed to nutrient insufficiency, the method comprising measuring the level of FGF21 or a fragment thereof in the sample obtained from a subject pregnant with the fetus, wherein a level of FGF21 in the sample elevated above control levels identifies a fetus exposed to nutrient insufficiency.

4. The method of claim 1, 2, or 3, wherein the FGF21 binding agent comprises an anti-FGF21 antibody.

5. A method of treating fetal growth restriction, the method comprising administering an amount of protein sufficient to reduce circulating FGF21 levels in a subject pregnant with a fetus determined to be afflicted with fetal growth restriction, wherein fetal growth restriction is determined by measuring FGF21 levels in a biological sample isolated from the pregnant mother.

6. A method of preventing fetal growth restriction, the method comprising administering a sufficient amount of a protein to a subject pregnant with a fetus at risk of fetal growth restriction to reduce circulating FGF21 levels, wherein risk of fetal growth restriction is determined by measuring FGF21 levels in a biological sample isolated from the pregnant mother.

7. The method of claim 5 or 6, wherein the measuring comprises incubating the biological sample with an agent that binds to FGF21 protein or a fragment thereof, wherein a FGF21-binding agent complex is formed.

8. The method of claim 7, wherein the FGF21-binding agent comprises an anti-FGF21 antibody.

9. The method of claim 1, 2, 3, 5, or 6, further comprising comparing the level of FGF21 protein in the sample to that of at least one control sample.

10. The method of claim 1, 2 or 3, further comprising administering to the pregnant mother an amount of protein sufficient to reduce circulating FGF21 levels.

11. The method of claim 5, 6, or 10, wherein the amount of protein sufficient to reduce circulating FGF21 levels comprises no less than about 0.88 g/kg of body weight/day.

12. The method of claim 1, 2, 3, 5, or 6, wherein the biological sample comprises serum, whole blood, plasma, or a combination thereof.

13. The method of claim 1, 2, 3, 5, or 6, wherein measuring comprises an immunoassay, a colorimetric assay, a fluorimetric assay or a combination thereof

14. The method of claim 13, wherein the immunoassay comprises a Western blot assay, an enzyme-linked immunosorbent assay (ELISA), immunoprecipitation or a combination thereof.

15. A method of treating fetal growth restriction, the method comprising administering to a subject pregnant with a fetus afflicted with or at risk of fetal growth restriction a therapeutically effective amount of an agent that reduces the circulating protein level of FGF21.

16. The method of claim 15, further comprising determining the risk for fetal growth restriction by measuring the level of FGF21 protein or a fragment thereof in a biological sample isolated from the subject.

17. The method of claim 15, wherein the agent is administered to the placenta.

18. The method of claim 15, wherein the agent comprises a synthetic polynucleotide that is targeted to the nucleic acid molecule encoding FGF21 (SEQ ID NO: 4).

19. The method of claim 18, wherein the synthetic polynucleotide is an siRNA.

20. The method of claim 1, 2, 3, or 7, wherein the FGF21 protein comprises SEQ ID NO:
3 or a fragment thereof.

21. The method of claim 1, 2, 3, or 7, wherein the FGF21 protein comprises SEQ ID NO: 6, or a fragment thereof

22. The method of claim 1, 2, 3, or 7, wherein the FGF21 fragment comprises N-terminal truncated FGF21 or C-terminal truncated FGF21.

23. The method of claim 21, wherein the N-terminal truncated FGF21 comprises SEQ ID NO: 5 or SEQ ID NO: 7.

24. A kit of molecular biomarkers for identifying a fetus at risk for growth restriction, the kit comprising at least one element for measuring the level of FGF21 protein or fragment thereof, and, optionally, at least one element for measuring the level of β-Klotho, where together represent a molecular signature that is indicative of fetal growth restriction.

25. The kit of claim 24, wherein the signature of fetal growth restriction comprises levels of FGF21 and, optionally, β-Klotho, above control levels.
